(19) United States
(12) Patent Application Publication
(10) Pub. No.: US 2006/0034943 A1
(43) Pub. Date: Feb. 16, 2006

(54) Process for treating a biological organism

(75) Inventor: Jack Tuszynski, Edmonton (CA)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: Technology Innovations LLC

(21) Appl. No.: 11/246,307

(22) Filed: Oct. 11, 2005

Related U.S. Application Data
(63) Continuation-in-part of application No. 10/976,274, filed on Oct. 28, 2004.
Application No. 10/976,274 is a continuation-in-part of application No. 10/923,615, filed on Aug. 20, 2004.
Application No. 10/976,274 is a continuation-in-part of application No. 10/808,618, filed on Mar. 24, 2004.
Application No. 10/976,274 is a continuation-in-part of application No. 10/867,517, filed on Jun. 14, 2004.
Application No. 10/976,274 is a continuation-in-part of application No. 10/878,905, filed on Jun. 28, 2004.
(60) Provisional application No. 60/516,134, filed on Oct. 31, 2003.

(51) Int. Cl. A61N 1/39; A61K 48/00; A61K 31/7048; A61K 31/704; A61K 31/7072

(52) U.S. Cl. 424/649; 514/34; 514/49; 514/251; 514/109; 514/27; 514/44; 514/575; 607/1

(57) ABSTRACT

A biological organism suffering from cancer can be treated by administering a cancer cell cycle arresting drug; optionally administering a microtubule stabilizing agent; and exposing the cell cycle arrested cells to mechanical vibrational energy. The method selectively induces apoptosis in cancer cells.

### CROSS-REFERENCE TO RELATED PATENT APPLICATIONS

This application claims priority from United States provisional patent application U.S. Ser. No. 60/516,134, filed on Oct. 31, 2003, the entire disclosure of which is hereby incorporated by reference into this specification.

This application is a continuation-in-part of applicants' U.S. patent application Ser. No. 10/976,274 (filed on Oct. 28, 2004), of applicants' U.S. patent application Ser. No. 10/923,615, (filed on Aug. 20, 2004), Ser. No. 10/808,618 (filed on Mar. 24, 2004), of applicants' U.S. patent application Ser. No. 10/867,517 (filed on Jun. 14, 2004), and of applicants' U.S. patent application Ser. No. 10/878,905 (filed on Jun. 28, 2004).

### FIELD OF THE INVENTION

A process for treating a biological organism in which a cell cycle arresting drug is administered to the organism to produce synchronized cells, optionally the microtubules within the synchronized cells are stabilized by means of a microtubule stabilizing agent, and the synchronized cells with the optionally stabilized microtubules are then contacted with mechanical vibrational energy, such as ultrasound energy.

### BACKGROUND OF THE INVENTION

Paclitaxel is a complex diterpenoid that is widely used as an anti-mitotic agent; it consists of a bulky, fused ring system and an extended side chain that is required for its activity. See, e.g., page 112 of Gunda I. Georg's “Taxane Anticancer Agents: Basic Science and Current Status,” ACS Symposium Series 583 (American Chemical Society, Washington, D.C., 1995).

The aqueous solubility of paclitaxel is relatively low. Thus, as is disclosed at page 112 of such Georg text, estimates of paclitaxel solubility vary widely, ranging from about 30 micrograms per milliliter and about 7 micrograms per milliliter to less than 0.7 micrograms per milliliter.

The molecular weight of paclitaxel is in excess of 700; this relatively high molecular weight is one factor that, according to the well-known “rule of 5,” contributes to paclitaxel poor water solubility.

The “rule of 5” was set forth by Christopher A. Lipinski et al. in an article entitled “Experimental and computational approaches to estimate solubility and permeability in drug discovery and development settings,” Adv. Drug Delivery Rev., 1997, 23(1-3), 3-25. In this article, it was disclosed that: “In the USAN set we found that the sum of Ns and Os in the molecular formula was greater than 10 in 12% of the compounds. Eleven percent of compounds had a MWT of over 500 . . . . The ‘rule of 5’ states that: poor absorption of permeation is more likely where: A. There are more than 5H-bond donors (expressed as the sum of OHs and NHs); B. The MWT is over 500; C. The LogP is over 500 . . . ; D. There are more than 10H-bond acceptors (expressed as the sum of Ns and Os).”

The Lipinski “rule of 5” has also erroneously been referred to as the “Pfizer rule of 5,” as is illustrated by U.S. Pat. No. 6,675,136, the entire disclosure of which is hereby incorporated by reference into this specification. As is disclosed in such patent, “To further illustrate the versatility of the present technique, we also introduce the concept of ‘anchor’ objects. Anchor objects are molecules situated at the corners of a region of the drug space that is defined by Pfizer's ‘rule of 5’. This rule has been empirically derived by a computer analysis of known drugs, as described by Christopher A. Pfizer and co-workers in Adv. Drug Delivery Rev., vol. 23, pp. 3-25 (1997). The ‘rule of 5” is focused on drug permeability and oral absorption . . . . According to Pfizer's “rule of 5”, LIPO and HBDON are between 0 and 5, HBACC is between 0 and 10, and M.W. has a maximum of 500.”

The problems that high molecular weight compounds have with poor water solubility are discussed in U.S. Pat. No. 6,667,048 of Karel J. Lambert et al., which discloses an “emulsion vehicle for a poorly soluble drug.” In the “background of the invention” section of this patent, it is disclosed that: “Hundreds of medically useful compounds are discovered every year, but clinical use of these drugs is possible only if a drug delivery vehicle is developed to transport them to their therapeutic target in the human body. This problem is particularly critical for drugs requiring intravenous injection in order to reach their therapeutic target or dosage but which are water insoluble or poorly water insoluble. For such hydrophobic compounds, direct injection may be impossible or highly dangerous, and can result in hemolysis, phlebitis, hypersensitivity, organ failure and/or death. Such compounds are termed by pharmacists ‘lipophilic,’ ‘hydrophobic,’ or in their most difficult form, ‘amphiphobic’

As is also disclosed in U.S. Pat. No. 6,667,048, “Administration of chemotherapeutic or anti-cancer agents is particularly problematic. Low solubility anti-cancer agents are difficult to solubilize and supply at therapeutically useful levels. On the other hand, water-soluble anti-cancer agents are generally taken up by both cancer and non-cancer cells thereby exhibiting non-specificity . . . . Efforts to improve water-solubility and comfort of administration of such agents have not solved, and may have worsened, the two fundamental problems of cancer chemotherapy: 1) non-specific toxicity, and 2) rapid clearance from the bloodstream by non-specific mechanisms. In the case of cytotoxins, which form the majority of currently available chemotherapies, these two problems are clearly related. Whenever the therapeutic is taken up by noncancerous cells, a diminished amount of the drug remains available to treat the cancer, and more importantly, the normal cell ingesting the drug is killed.”

As is also disclosed in U.S. Pat. No. 6,667,048, “The chemotherapeutic must be present throughout the affected tissue(s) at high concentration for a sustained period of time so that it may be taken up by the cancer cells, but not at so high a concentration that normal cells are injured beyond repair. Obviously, water-soluble molecules can be administered in this way, but only by slow, continuous infusion and monitoring, aspects which entail great difficulty, expense and inconvenience.”

It does not appear that the prior art has provided a water-soluble anti-mitotic agent that is capable of solving the problems discussed in U.S. Pat. No. 6,667,048. It is an object of this invention to provide such an agent. In particular, and in one embodiment, it is an object of this invention to provide a magnetic anti-mitotic composition that can be directed to be more toxic to cancer cells than normal cells. Furthermore, and in another embodiment, it is another object of this invention to provide a delivery system that will provide a chemotherapeutic agent at a high concentration for a sustained period of time but not at such a high concentration that a substantial number of normal cells are injured beyond repair.

It is yet another object of this invention to provide a process for treating a biological organism in which the water soluble anti-mitotic agent may be used to both synchronize certain cells and immobilize the microtubules within such cells.

### SUMMARY OF THE INVENTION

In accordance with one embodiment of this invention, there is provided a process for treating a biological organism in which a cell cycle arresting drug is administered to the organism to produce synchronized cells, optionally the microtubules within the synchronized cells are stabilized by means of a microtubule stabilizing agent, and the synchronized cells with the optionally stabilized microtubules are then contacted with mechanical vibrational energy.

### BRIEF DESCRIPTION OF THE DRAWING FIGURES

The invention will be described with reference to the specification and the enclosed drawings, in which like numerals refer to like elements, and wherein:

FIG. 1 is a schematic illustration of one preferred implantable assembly of the invention;

FIG. 2 is a schematic illustration of a flow meter that may be used in conjunction with the implantable assembly of claim 1;

FIG. 3 is a flow diagram of one preferred process of the invention;

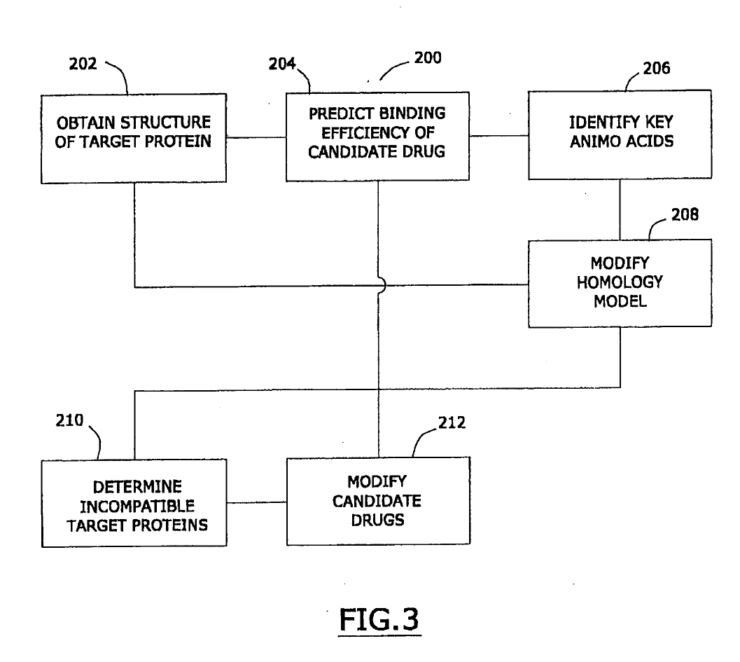

FIG. 4 is a flow diagram of another preferred process of the invention; and

FIG. 5 is a flow diagram of yet another preferred process of the invention.

### DESCRIPTION OF THE PREFERRED EMBODIMENTS

The present invention includes magnetic anti-mitotic compounds that bind tubulin and/or microtubules and/or various proteins involved in microtubule dynamics.

The invention also provides a process for treating a biological organism in which a magnetic anti-mitotic compound is used to both synchronize certain cells and immobilize the microtubules within such cells prior to the time such cells are subjected to mechanical vibrational energy.

Microtubules are extremely important in the process of mitosis. Their importance in mitosis and cell division makes microtubules an important target for anticancer drugs. M. A. Jordan et al., “Microtubules as a target for anticancer drugs”, Nature Reviews/Cancer, 4, pages 253-266, April 2004 (incorporated herein by reference). Microtubules and their dynamics are the targets of a chemically diverse group of antimitotic drugs (with various tubulin-binding sites) that have been used with great success in the treatment of cancer. Id. at 253.

Microtubule dynamics are crucial to mitosis. Id at 255. Many compounds useful in cancer therapy exert a cytotoxic effect by disrupting or interacting with microtubules, e.g., Vinblastine (Velban), Vincristine (Oncovin); Vinorelbine (Navelbine), Vinflnine, cryptophycin 52, the halichondrins (such as, e.g., E7389), the dolastatins (such as TZT-1027), the hemiasterlins (such as HTI-286), colchicine, the combretastatins (AVE8062A, CA-1-P, CA-4-P, N-acetylcolchicinol-O-phosphate, ZD6126), the methoxybenzene-sulphonamides (such as ABT-751, E7010, etc.), taxanes (such as paclitaxel (Taxol™), docetaxel (Taxotere™), the epothilones (such as BMS-247550, epothilones B and D), estramustine, and others.

Anti-mitotic drugs interfere with these “microtubule dynamics” in different ways. A large number of chemically diverse substances bind to soluble tubulin and/or directly to tubulin in the microtubules. Jordan et al. at 257.

In one embodiment of the invention, magnetic anti-mitotic drugs bind directly to soluble tubulin. In another embodiment, magnetic anti-mitotic drugs bind to the polymerized tubulin in the microtubules. In yet another embodiment, magnetic anti-mitotic compounds act on the polymerization dynamics of the spindle microtubules, e.g., by inhibiting or stimulating polymerization. Still another embodiment involves antimitotic compounds slowing or blocking mitosis at the metaphase-anaphase transition.

In one embodiment of this invention, the antimitotic compounds of this invention inhibit the process of angiogenesis (the formation of new blood vessels). In another embodiment of this invention, the antimitotic compounds of this invention shut down the existing vasculature of tumors. Compositions having these antivascular effects have been reported. For example, microtubule-targeting agents can shut down existing tumor vasculature. See G. M. Tozer et al., “The biology of the combretastatins as tumor vascular targeting agents,” Int. J. Exp. Pathol., 83: 21-38 (2002) (incorporated herein by reference).

Vascular-targeting agents can damage tumour vasculature without significantly harming normal tissues. V. E. Prise et al., “The vascular response of tumor and normal tissues in the rat to the vascular targeting agent combrestatin A4 phosphate, at clinically relevant doses,” Int. J. Oncol. 21: 717-726 (2002). In one embodiment, the magnetic anti-mitotic compound of this invention damages tumors without significantly harming normal tissues.

The source of this specificity is not known, but has been suggested to be attributable to differences between the mature vasculature of normal tissues and the immature or forming vasculature of tumors. There are suggestions that endothelial cells of immature vasculature could have a less well-developed actin cytoskeleton that might make the cells more susceptible to collapse. P. D. Davis et al., “ZD6126: A novel vascular-targeting agent that causes selective destruction of tumor vasculature,” Cancer Res. 62: 7247-7253 (2003) (incorporated herein by reference).

In one preferred embodiment of this invention, magnetic anti-mitotic compounds of this invention bind to, and inactivate, a tubulin isotype that causes, or tends to cause, drug-resistance.

Almost all eukaryotic cells contain microtubules which comprise a major component of the network of proteinaceous filaments known as the cytoskeleton. Microtubules thereby participate in the control of cell shape and intracellular transport. They are also the principal constituent of mitotic and meiotic spindles, cilia and flagella. In plants, microtubules have additional specialized roles in cell division and cell expansion during development. U.S. Pat. No. 5,888,818, which is incorporated herein by reference.

As is also disclosed in U.S. Pat. No. 5,888,818, “In terms of their composition, microtubules are proteinaceous hollow rods with a diameter of approximately 24 nm and highly variable length. They are assembled from heterodimer subunits of an α-tubulin and a β-tubulin polypeptide, each with a molecular weight of approximately 50,000. Both polypeptides are highly flexible globular proteins (approximately 445 amino acids), each with a predicted 25% α-helical and 40% β-pleated sheet content. In addition to the two major forms (α- and β-tubulin), there is a rare δ-tubulin form which does not appear to participate directly in the formation of microtubule structure, but rather it may function in the initiation of microtubule structure.”

In one embodiment of this invention, the magnetic anti-mitotic agent of this invention binds to a target site on a β-tubulin polypeptide. In another embodiment of this invention, the anti-mitotic compounds selectively covalently modify β-tubulin isotypes but does not covalently modify other proteins.

Methods of preparing paclitaxel and its analogues and derivatives are well-known in the art, and are described, for example, in U.S. Pat. Nos. 5,569,729; 5,565,478; 5,530,020; 5,527,924; 5,484,809; 5,475,120; 5,440,057; and 5,296,506. Paclitaxel and its analogues and derivatives are also available commercially. Paclitaxel, for example, can be obtained from Bristol-Myers Squibb Company, Oncology Division (Princeton, N.J.), under the registered trademark Taxol®.

The methods of the present invention may be used to treat neoplasia in a subject in need of treatment. Neoplasias for which the present invention will be particularly useful include, without limitation, carcinomas, particularly those of the bladder, breast, cervix, colon, head, kidney, lung, neck, ovary, prostate, and stomach; lymphocytic leukemias, particularly acute lymphoblastic leukemia and chronic lymphocytic leukemia; myeloid leukemias, particularly acute monocytic leukemia, acute promyelocytic leukemia, and chronic myelocytic leukemia; malignant lymphomas, particularly Burkitt's lymphoma and Non-Hodgkin's lymphoma; malignant melanomas; myeloproliferative diseases; sarcomas, particularly Ewing's sarcoma, hemangiosarcoma, Kaposi's sarcoma, liposarcoma, peripheral neuroepithelioma, and synovial sarcoma; and mixed types of neoplasias, particularly carcinosarcoma and Hodgkin's disease.

Discodermolide also may provide a means to circumvent clinical resistance due to overproduction of P-glycoprotein. Accordingly, the combination of paclitaxel and discodermolide may be advantageous for use in subjects who exhibit resistance to paclitaxel (Taxol®). See, e.g., U.S. Pat. No. 6,541,509 (incorporated herein by reference).

In the method of the present invention, administration of paclitaxel in combination with other antineoplastic agents, e.g., discodermolide, refers to co-administration of the two antineoplastic agents. Co-administration can occur concurrently, sequentially, or alternately. Paclitaxel and other antineoplastic agents, e.g., discodermolide, can also be co-administered to a subject in separate, individual formulations that are spaced out over a period of time, so as to obtain the maximum efficacy of the combination. Administration of each drug may range in duration from a brief, rapid administration to a continuous perfusion. When spaced out over a period of time, co-administration of paclitaxel and discodermolide may be sequential or alternate. For sequential co-administration, one of the antineoplastic agents is separately administered, followed by the other. For example, a full course of treatment with paclitaxel may be completed, and then may be followed by a full course of treatment with discodermolide. Alternatively, for sequential co-administration, a full course of treatment with discodermolide may be completed, then followed by a full course of treatment with paclitaxel. For alternate co-administration, partial courses of treatment with paclitaxel may be alternated with partial courses of treatment with discodermolide, until a full treatment of each drug has been administered.

The effective antineoplastic amount of paclitaxel (Taxol®) administered intraperitoneally may range from 1 to 10 mg/kg, and doses administered intravenously may range from 1 to 3 mg/kg, or from 135 mg/m2 to 200 mg/m2. The appropriate effective antineoplastic amounts of paclitaxel can be readily determined by the skilled artisan.

Preferred Anti-Mitotic Compounds

In this section of the specification, a preferred compound is discussed. The preferred compound of this embodiment of the invention is an anti-mitotic compound. Anti-mitotic compounds are known to those skilled in the art. Reference may be had, e.g., to U.S. Pat. Nos. 6,723,858 (estrogenic compounds as anti-mitotic agents), 6,528,676 (estrogenic compounds as anti-mitotic agents), U.S. Pat. No. 6,350,777 (anti-mitotic agents which inhibit tubulin polymerization), 6,162,930 (anti-mitotic agents which inhibit tubulin polymerization), 5,892,069 (estrogenic compounds as anti-mitotic agents), 5,886,025 (anti-mitotic agents which inhibit tubulin polymerization), 5,661,143 (estrogenic compounds as anti-mitotic agents), 3,997,506 (anti-mitotic derivatives of thiocolchicine), and the like. The entire disclosure of each of these United States patent Nos. is hereby incorporated by reference into this specification.

These prior art anti-mitotic agents may be modified, in accordance with the process of this invention, to make them “magnetic,” as that term is defined in this specification. In the next section of this specification, processes for modifying taxanes to make them “magnetic” are described. Analogous methods can be used to make other anti-mitotic compounds magnetic, e.g., colchicine and the vinca alkaloids, and such compounds can also be used to treat patients suffering from, or at risk of, cancer. Any one or more of such magnetic anti-mitotic agents can also be conjointly administered with a conventional (non-magnetic) anti-mitotic agent to achieve the desired antitumor, tumoricidal, or cytotoxic effect.

Preparation and Use of Magnetic Taxanes

This section describes the preparation of certain magnetic taxanes that may be used in one or more of the processes of his invention. The process that is used to make such taxanes magnetic and/or water soluble may also be used to make other anti-mitotic compounds magnetic and/or water soluble.

In one embodiment of the invention, a biologically active substrate or agent is linked to a magnetic carrier particle. An external magnetic field may then be used to increase the concentration of a magnetically linked drug at a predetermined location.

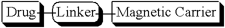

One method for the introduction of a magnetic carrier particle involves the linking of a drug with a magnetic carrier. While some naturally occurring drugs inherently carry magnetic particles (ferrimycin, albomycin, salmycin, etc.), it is more common to generate a synthetic analog of the target drug and attach the magnetic carrier through a linker.

Functionalized Taxanes

Paclitaxel and docetaxel are members of the taxane family of compounds. In one embodiment of the invention, such a linker is covalently attached to at least one of the positions in a taxane.

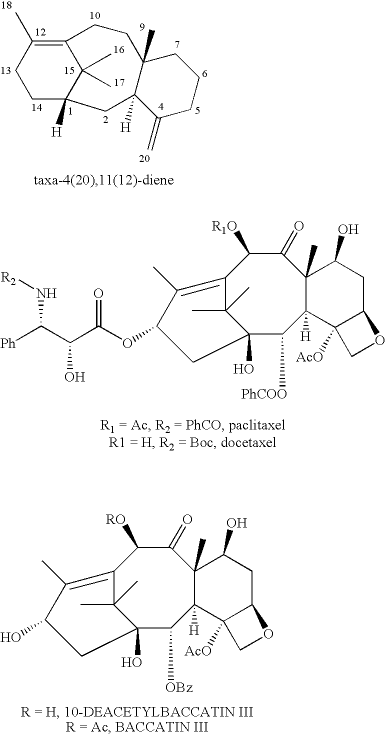

The northern hemisphere of taxanes has been altered without significant impact on the biological activity of the drug. See, e.g., Taxane Anticancer Agents, Basic Science and Current Status, G. George et al., eds., ACS Symposium Series 583, 207th National Meeting of the American Chemical Society, Chapter 15, San Diego, Calif. (1994).

Specifically, the C-7, C-9, and C-10 positions of paclitaxel have been significantly altered without degrading the biological activity of the parent compound. Likewise the C-4 position appears to play only a minor role. The oxetane ring at C-4 to C-5 has been shown to be critical to biological activity. Likewise, certain functional groups on the C-13 side chain have been shown to be of particular importance.

In one embodiment of the invention, a position within paclitaxel is functionalized to link a magnetic carrier particle. A number of suitable positions are presented below. It should be understood that paclitaxel is illustrated in the figures below, but other taxane analogs may also be employed. 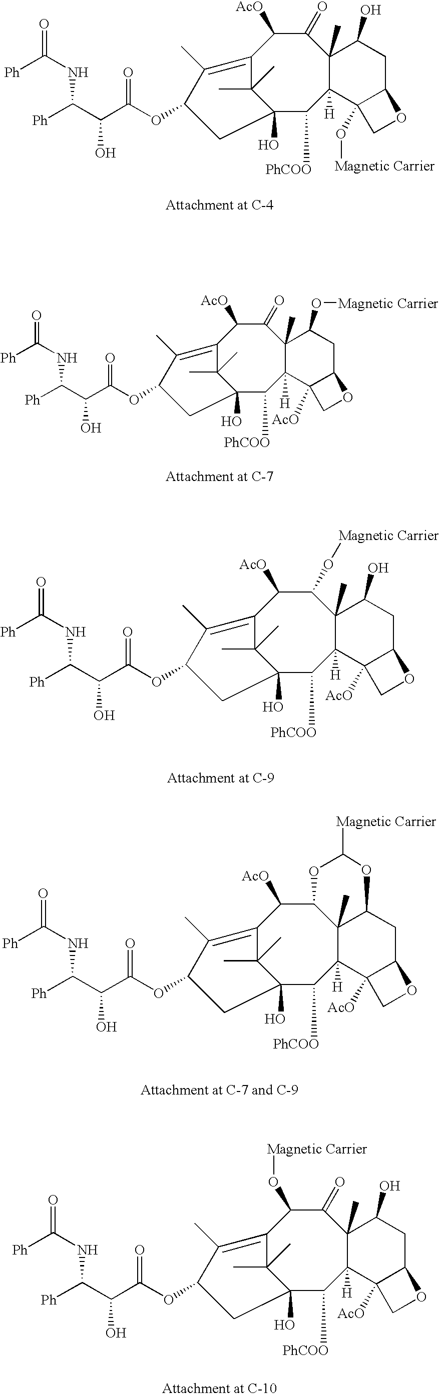 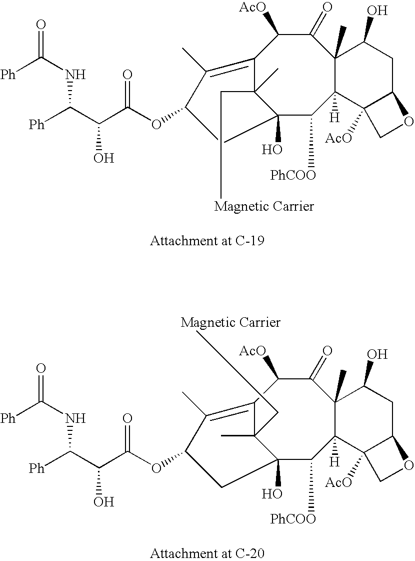
Attachment at C-4
C-4 taxane analogs have been previously generated in the art. A wide range of methodologies exist for the introduction of a variety of substituents at the C-4 position. By way of illustration, reference may be had to “Synthesis and Biological Evaluation of Novel C-4 Aziridine-Bearing Paclitaxel Analogs” by S. Chen et al., J. Med. Chem. 1995, vol 38, pp 2263.

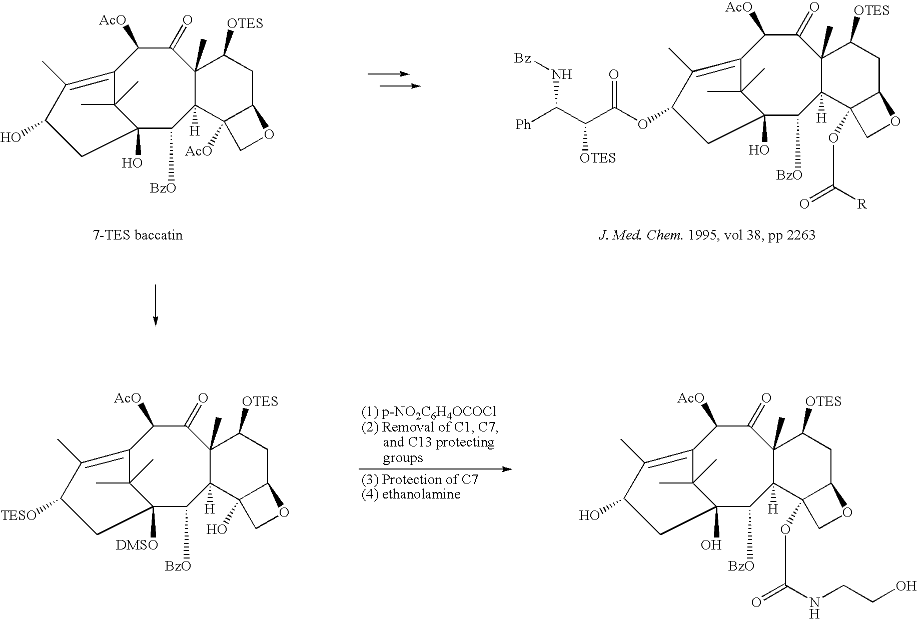

The secondary (C-13) and tertiary (C-1) alcohols of 7-TES baccatin were protected using the procedure of Chen (J. Org. Chem. 1994, vol 59, p 6156) while simultaneously unmasking the alcohol at C-4. The resulting product was treated with a chloroformate to yield the corresponding carboxylate. Removal of the silyl protecting groups at C-1, C-7, and C-13, followed by selective re-protection of the C-7 position gave the desired activated carboxylate. The compound was then treated with a suitable nucleophile (in the author's case, ethanolamine) to produce a C-4 functionalized taxane. The C-13 side chain was installed using standard lactam methodology.

This synthetic scheme thus provides access to a variety of C-4 taxane analogs by simply altering the nucleophile used. In one embodiment of the instant invention, the nucleophile is selected so as to allow the attachment of a magnetic carrier to the C-4 position.

Attachment at C-7

The C-7 position is readily accessed by the procedures taught in U.S. Pat. No. 6,610,860. The alcohol at the C-10 position of 10-deacetylbaccatin III was selectively protected. The resulting product was then allowed to react with an acid halide to produce the corresponding ester by selectively acylating the C-7 position over the C-13 alcohol. Standard lactam methodology allowed the installation of the C-13 side chain. In another embodiment, baccatin III, as opposed to its deacylated analog, is used as the starting material.

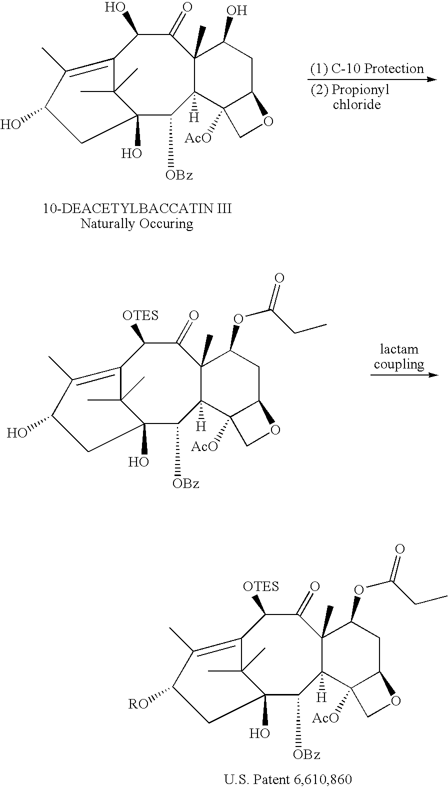

Other C-7 taxane analogs are disclosed in U.S. Pat. Nos. 6,610,860; 6,359,154; and 6,673,833, the contents of which are hereby incorporated by reference.

Attachment at C-9

It has been established that the C-9 carbonyl of paclitaxel is relatively chemically inaccessible, although there are exceptions (see, for example, Tetrahedron Lett. Vol 35, p 4999). However, scientists gained access to C-9 analogs when 13-acetyl-9-dihydrobaccatin III was isolated from Taxus candidensis (see J. Nat. Products, 1992, vol 55, p 55 and Tetrahedron Lett. 1992, vol 33, p 5173). This triol is currently used to provide access to a variety of such C-9 analogues.

In chapter 20 of Taxane Anticancer Agents, Basic Science and Current Status, (edited by G. George et al., ACS Symposium Series 583, 207th National Meeting of the American Chemical Society, San Diego, Calif. (1994)) Klein describes a number of C-7/C-9 taxane analogs. One of routes discussed by Klein begins with the selective deacylation of 13-acetyl-9-dihydrobaccatin III, followed by the selective protection of the C7 alcohol as the silyl ether. A standard lactam coupling introduced the C-13 side chain. The alcohols at C-7 and C-9 were sufficiently differentiated to allow a wide range of analogs to be generated. “In contrast to the sensitivity of the C-9 carbonyl series under basic conditions, the 9(R)-dihydro system can be treated directly with strong base in order to alkylate the C-7 and/or the C-9 hydroxyl groups.”

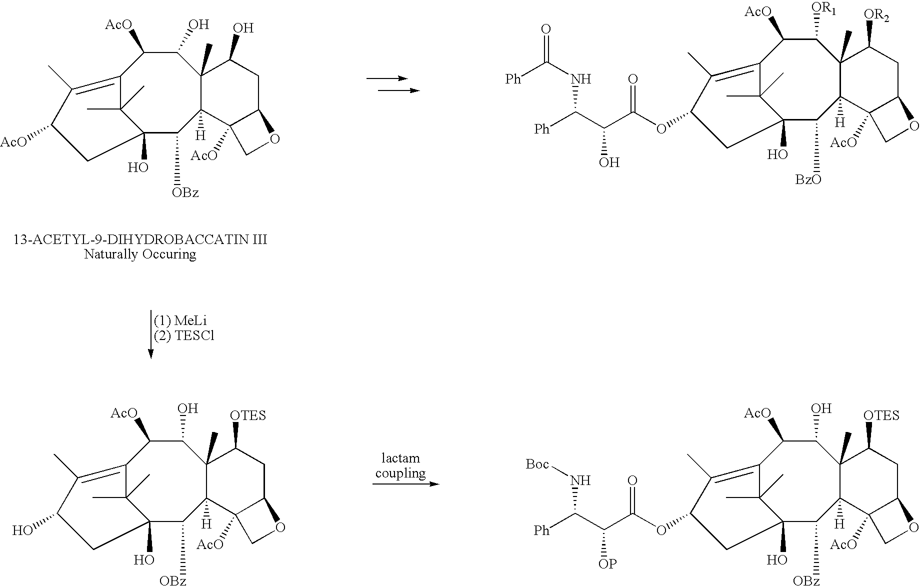

One skilled in the art may adapt Klein's general procedures to install a variety of magnetic carriers at these positions. Such minor adaptations are routine for those skilled in the art.

Attachment at C-7 and C-9

Klein also describes a procedure wherein 13-acetyl-9-dihydrobaccatin III is converted to 9-dihydrotaxol. Reference may be had to “Synthesis of 9-Dihydrotaxol: a Novel Bioactive Taxane” by L. L. Klein in Tetrahedron Lett. Vol 34, pp 2047-2050. An intermediate in this synthetic pathway is the dimethylketal of 9-dihydrotaxol.

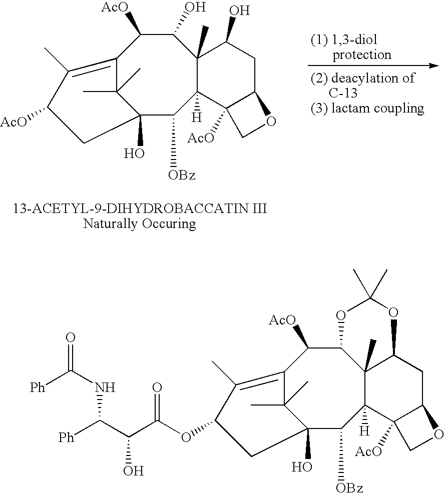

In one embodiment, the procedure of Klein is followed with a carbonyl compound other than acetone to bind a wide variety of groups to the subject ketal. Supplemental discussion of C-9 analogs is found in “Synthesis of 9-Deoxotaxane Analogs” by L. L. Klein in Tetrahedron Lett. Vol 35, p 4707 (1994).

Attachment at C-10

In one embodiment of the invention, the C-10 position is functionalized using the procedure disclosed in U.S. Pat. No. 6,638,973. This patent teaches the synthesis of paclitaxel analogs that vary at the C-10 position. A sample of 10-deacetylbaccatin III was acylated by treatment with propionic anhydride. The C-13 side chain was attached using standard lactam methodology after first performing a selective protection of the secondary alcohol at the C-7 position. In one embodiment of the invention, this procedure is adapted to allow access to a variety of C-10 analogues of paclitaxel.

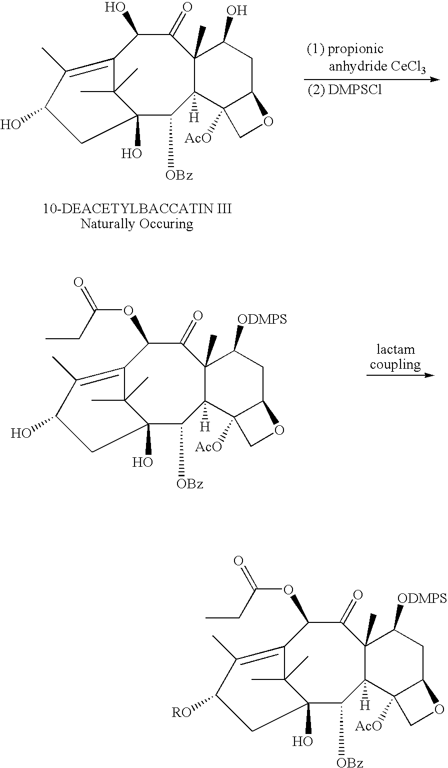

In one embodiment an anhydride is used as an electrophile. In another embodiment, an acid halide is used. As would be apparent to one of ordinary skill in the art, a variety of electrophiles could be employed. 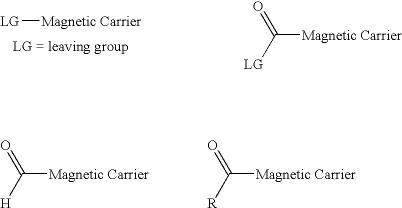
Siderophores

In one embodiment, a member of the taxane family of compounds is attached to a magnetic carrier particle. Suitable carrier particles include siderophores (both iron and non-iron containing), nitroxides, as well as other magnetic carriers.
Siderophores are a class of compounds that act as chelating agents for various metals. Most organisms use siderophores to chelate iron (III) although other metals may be exchanged for iron (see, for example, Exchange of Iron by Gallium in Siderophores by Energy, Biochemistry 1986, vol 25, pages 4629-4633). Most of the siderophores known to date are either catecholates or hydroxamic acids.

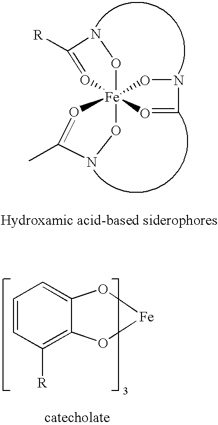

Representative examples of catecholate siderophores include the albomycins, agrobactin, parabactin, enterobactin, and the like.

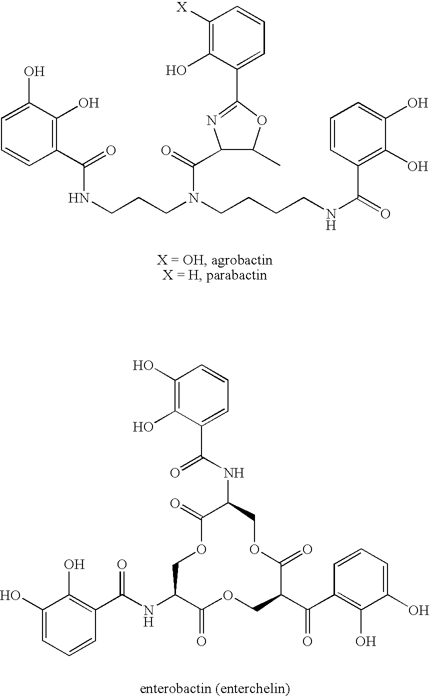

Examples of hydroxamic acid-based siderophores include ferrichrome, ferricrocin, the albomycins, ferrioxamines, rhodotorulic acid, and the like. Reference may be had to Microbial Iron Chelators as Drug Delivery Agents by M. J. Miller et al., Acc. Chem. Res. 1993, vol 26, pp 241-249; Structure of Des(diserylglycyl)ferrirhodin, DDF, a Novel Siderophore from Aspergillus ochraceous by M. A. F. Jalal et al., J. Org. Chem. 1985, vol 50, pp5642-5645; Synthesis and Solution Structure of Microbial Siderophores by R. J. Bergeron, Chem. Rev. 1984, vol 84, pp 587-602; and Coordination Chemistry and Microbial Iron Transport by K. N. Raymond, Acc. Chem. Res., 1979, vol 12, pp 183-190. The synthesis of a retrohydroxamate analog of ferrichrome is described by R. K. Olsen et al. in J. Org. Chem. 1985, vol 50, pp 2264-2271.

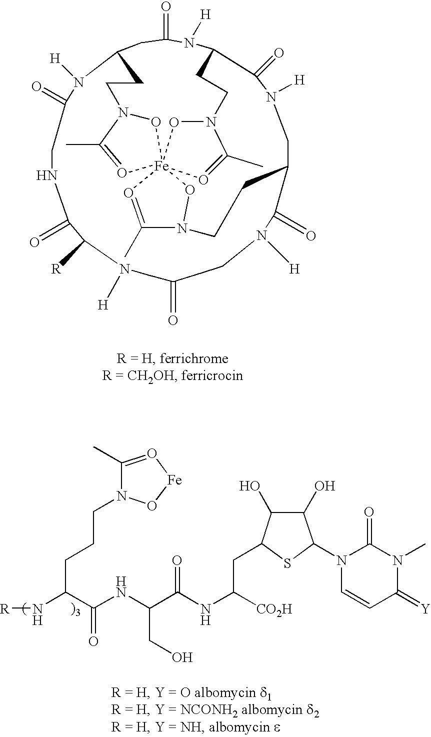

In “Total Synthesis of Desferrisalmycin” (M. J. Miller et al. in J. Am. Chem. Soc. 2002, vol 124 pp 15001-15005), a natural product is synthesized that contains a siderophore. The author states “siderophores are functionally defined as low molecular mass molecules which acquire iron (III) from the environment and transport it into microorganisms. Because of the significant roles they play in the active transport of physiologically essentially iron (III) through microbe cell members, it is not surprising that siderophores-drug conjugates are attracting more and more attention from both medicinal chemists and clinical researchers as novel drug delivery systems in the war against microbial infections, especially in an area of widespread emergency of multidrug-resistance (MDR) strains. There have been three families of compounds identified as natural siderophore-drug conjugates, including ferrimycin, albomycin, and salmycin.” In a related paper, Miller describes the use of siderophores as drug delivery agents (Acc. Chem. Res. 1993, vol 26, pp 241-249. Presumably, the siderophore acts as a “sequestering agents [to] facilitate the active transport of chelated iron into cells where, by modification, reduction, or siderophore decomposition, it is released for use by the cell.” Miller describes the process of tethering a drug to a Siderophore to promote the active transport of the drug across the cell membrane.

In “The Preparation of a Fully Differentiated ‘Multiwarhead’ Siderophore Precursor”, by M. J. Miller et al (J. Org. Chem. 2003, vol 68, pp 191-194) a precursor is disclosed which allows for a drug to be tethered to a Siderophore. In one embodiment, the route disclosed by Miller is employed to provide a variety of siderophores of similar structure. The synthesis of similar hydroxamic acid-based siderophores is discussed in J. Org. Chem. 2000, vol 65 (Total Synthesis of the Siderophore Danoxamine by M. J. Miller et al.), pp 4833-4838 and in the J. of Med. Chem. 1991, vol 32, pp 968-978 (by M. J. Miller et al.).

A variety of fluorescent labels have been attached to ferrichrome analogues in “Modular Fluorescent-Labeled Siderophore Analogues” by A. Shanzer et al. in J. Med. Chem. 1998, vol 41, 1671-1678. The authors have developed a general methodology for such attachments.

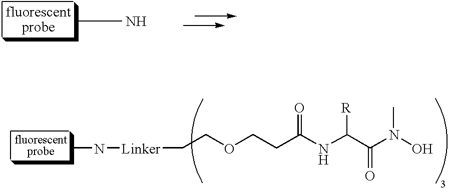

As discussed above, functionalized ferrichrome analogs have been previous generated, usually using basic amine acids (glycine). In one embodiment, functionality is introduced using an alternative amine acid (such as serine) in place of the central glycine residue. This provides a functional group foothold from which to base a wide variety of analogs. Using traditional synthetic techniques, various linkers are utilized so as to increase or decrease the distance between the magnetic carrier and the drug.

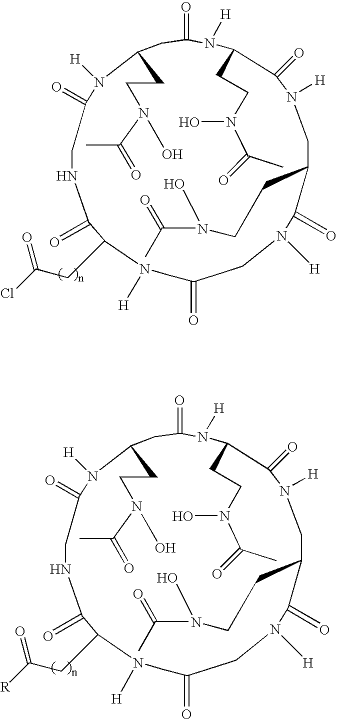

As would be apparent to one of ordinary skill in the art, the above specified techniques are widely applicable to a variety of substrates. By way of illustration, and not limitation, a number of magnetic taxanes are shown below. 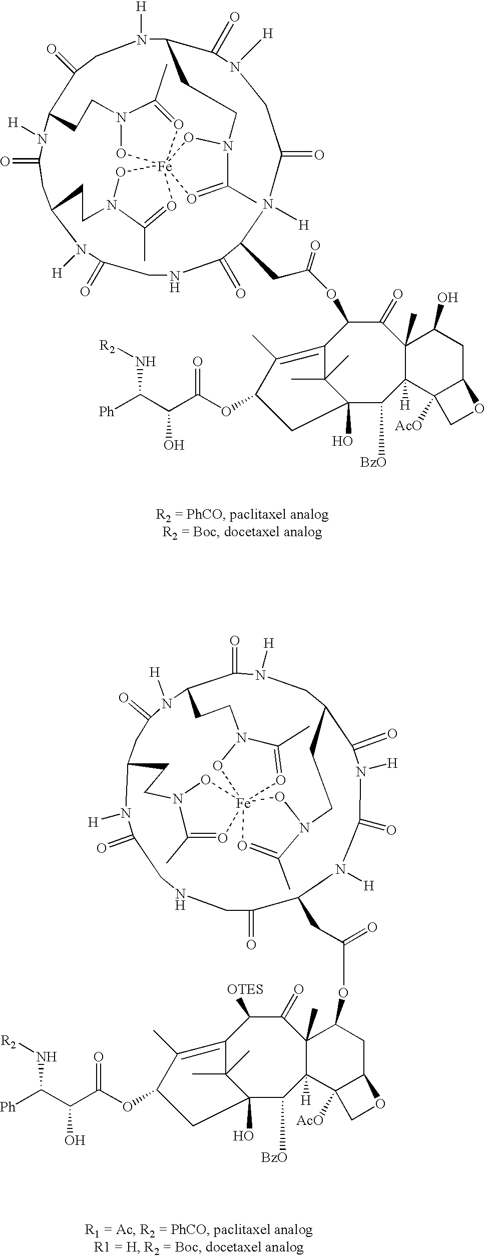 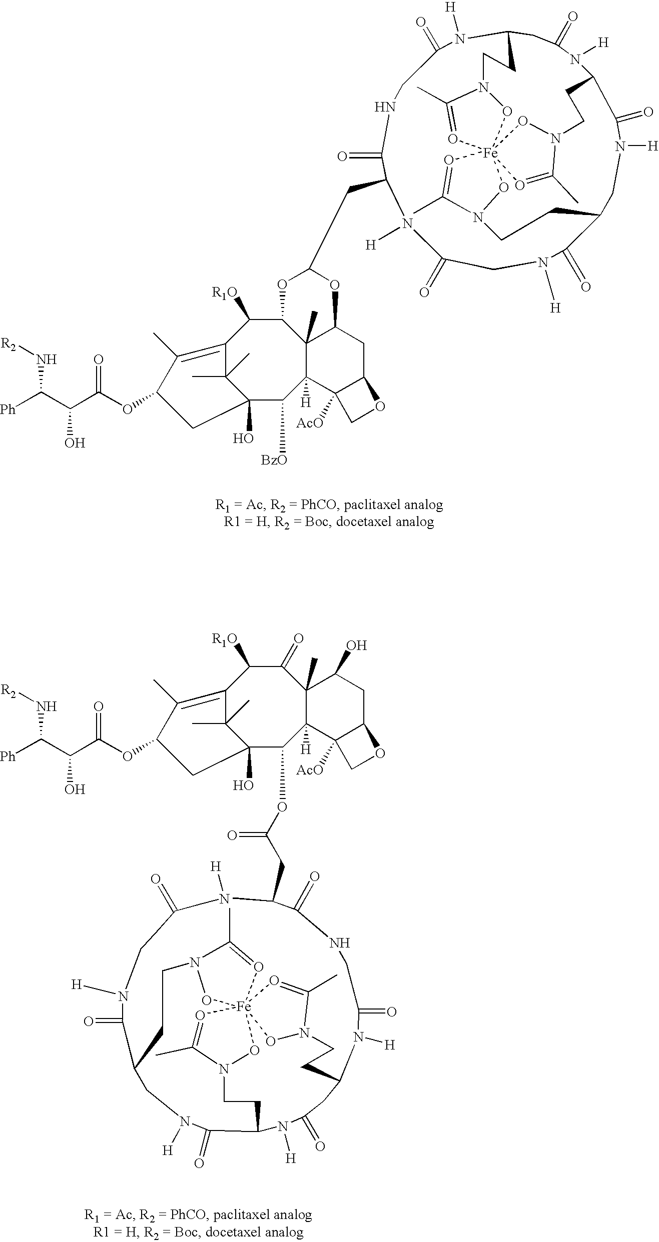
Nitroxides

Another class of magnetic carriers is the nitroxyl radicals (also known as nitroxides). Nitroxyl radicals a “persistent” radials that are unusually stable. A wide variety of nitroxyls are commercially available. Their paramagnetic nature allows them to be used a spin labels and spin probes.

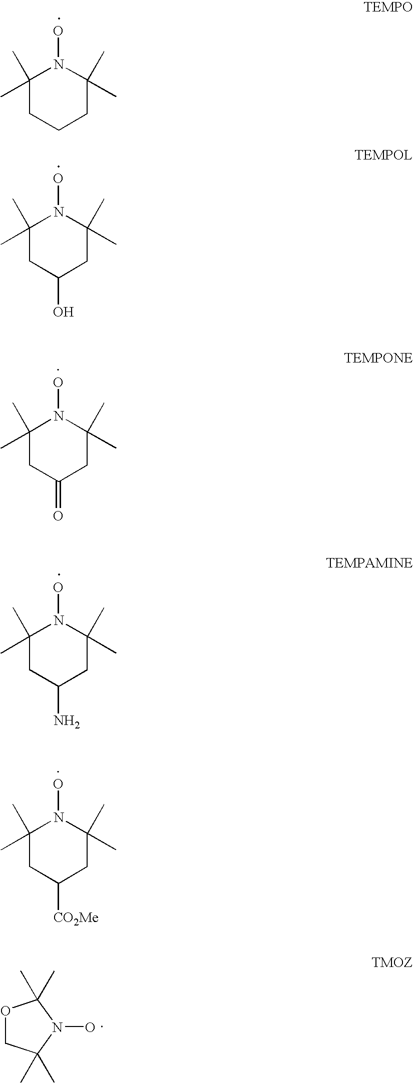

In addition to the commercially available nitroxyls, other paramagnetic radical labels have be generated by acid catalyzed condensation with 2-Amino-2-methyl-1-propanol followed by oxidation of the amine.

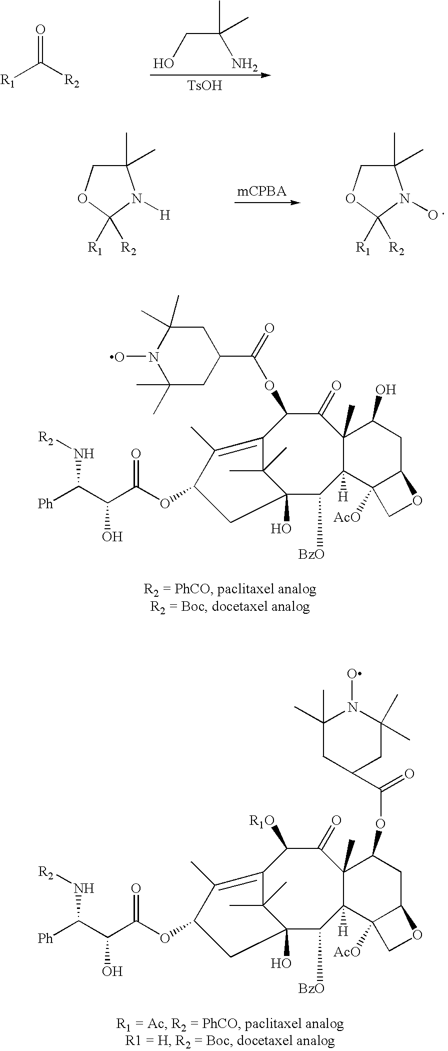

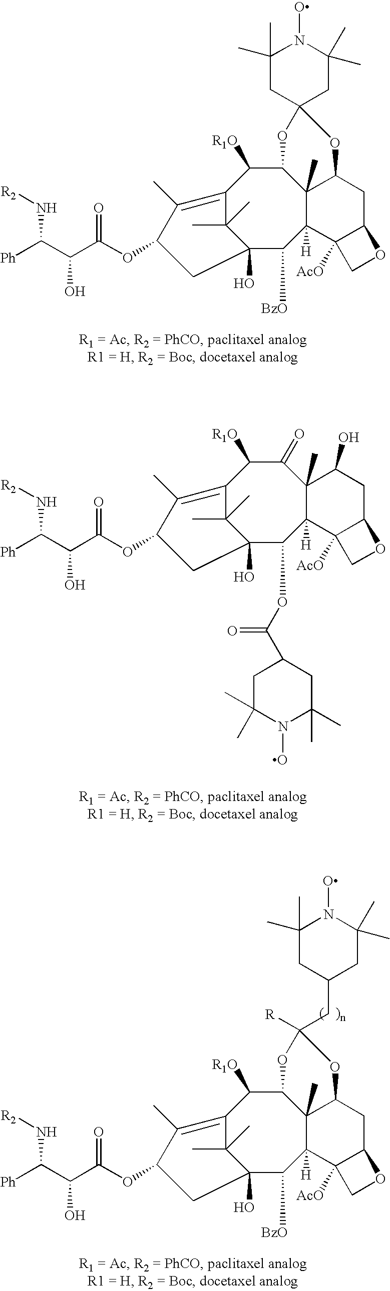

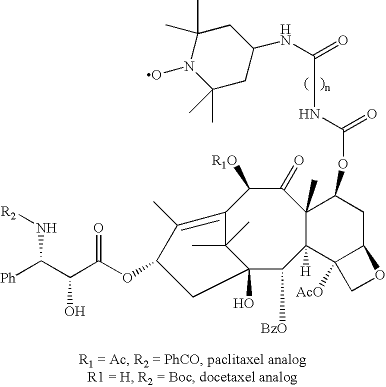

One of ordinary skill in the art could use the teachings of this specification to generate a wide variety of suitable carrier-drug complexes. The following table represents but a small sampling of such compounds.

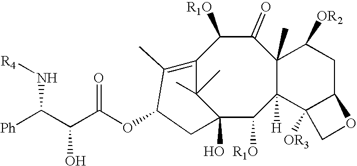

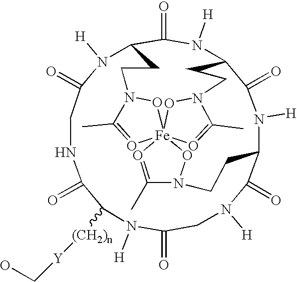

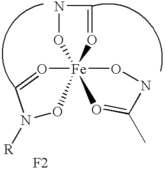

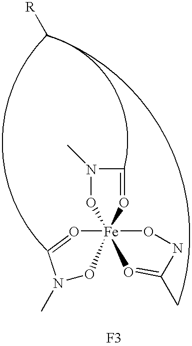

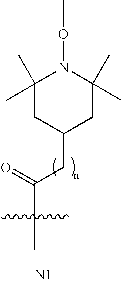

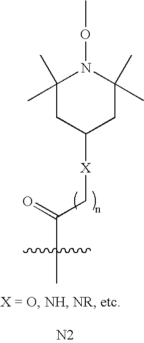

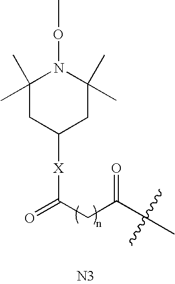

| R1 | R2 | R3 | R4 |
|---|---|---|---|
| F1, Y = CH2, | H | Ac | COPh |
| n = 0 to 20 | | | |
| Ac | F1, Y = CH2, | Ac | COPh |
| | n = 0 to 20 | | |
| Ac | H | F1, Y = CH2, | COPh |
| | | n = 0 to 20 | |
| Ac | H | Ac | F1, Y = CH2, |
| | | | n = 0 to 20 |
| H | H | Ac | Boc |
| F1, Y = CH2, | H | Ac | Boc |
| n = 0 to 20 | | | |
| H | F1, Y = CH2, | Ac | Boc |
| | n = 0 to 20 | | |
| H | H | F1, Y = CH2, | Boc |
| | | n = 0 to 20 | |
| H | H | Ac | F1, Y = CH2, |
| | | | n = 0 to 20 |
| F1, Y = NH | H | Ac | COPh |
| or NR, n = 0 | | | |
| to 20 | | | |
| Ac | F1, Y = NH | Ac | COPh |
| | or NR, n = 0 | | |
| | to 20 | | |
| Ac | H | F1, Y = NH | COPh |
| | | or NR, n = 0 | |
| | | to 20 | |
| Ac | H | Ac | F1, Y = NH |
| | | | or NR, n = 0 |
| | | | to 20 |
| H | H | Ac | Boc |
| F1, Y = NH | H | Ac | Boc |
| or NR, n = 0 | | | |
| to 20 | | | |
| H | F1, Y = NH | Ac | Boc |
| | or NR, n = 0 | | |
| | to 20 | | |
| H | H | F1, Y = NH | Boc |
| | | or NR, n = 0 | |
| | | to 20 | |
| H | H | Ac | F1, Y = NH |
| | | | or NR, n = 0 |
| | | | to 20 |
| N1, n = 0 to 20 | H | Ac | COPh |
| Ac | N1, n = 0 to 20 | Ac | COPh |
| Ac | H | N1, n = 0 to 20 | COPh |
| Ac | H | Ac | N1, n = 0 to 20 |
| H | H | Ac | Boc |
| N1, n = 0 to 20 | H | Ac | Boc |
| H | N1, n = 0 to 20 | Ac | Boc |
| H | H | N1, n = 0 to 20 | Boc |
| H | H | Ac | N1, n = 0 to 20 |
| N2, n = 0 to | H | Ac | COPh |
| 20, X = O or | | | |
| NH | | | |
| Ac | N2, n = 0 to | Ac | COPh |
| | 20, X = O or | | |
| | NH | | |
| Ac | H | N2, n = 0 to | COPh |
| | | 20, X = O or | |
| | | NH | |
| Ac | H | Ac | N2, n = 0 to |
| | | | 20, X = O or |
| | | | NH |
| H | H | Ac | Boc |
| N2, n = 0 to | H | Ac | Boc |
| 20, X = O or | | | |
| NH | | | |
| H | N2, n = 0 to | Ac | Boc |
| | 20, X = O or | | |
| | NH | | |
| H | H | N2, n = 0 to | Boc |
| | | 20, X = O or | |
| | | NH | |
| H | H | Ac | N2, n = 0 to |
| | | | 20, X = O or |
| | | | NH |
| N3, n = 0 to | H | Ac | COPh |
| 20, X = O or | | | |
| NH | | | |
| Ac | N3, n = 0 to | Ac | COPh |
| | 20, X = O or | | |
| | NH | | |
| Ac | H | N3, n = 0 to | COPh |
| | | 20, X = O or | |
| | | NH | |
| Ac | H | Ac | N3, n = 0 to |
| | | | 20, X = O or |
| | | | NH |
| H | H | Ac | Boc |
| N3, n = 0 to | H | Ac | Boc |
| 20, X = O or | | | |
| NH | | | |
| H | N2, n = 0 to | Ac | Boc |
| | 20, X = O or | | |
| | NH | | |
| H | H | N2, n = 0 to | Boc |
| | | 20, X = O or | |
| | | NH | |
| H | H | Ac | N3, n = 0 to |
| | | | 20, X = O or |
| | | | NH |
| F2 or F3 | H | Ac | COPh |
| Ac | F2 or F3 | Ac | COPh |
| Ac | H | F2 or F3 | COPh |
| Ac | H | Ac | F2 or F3 |
| F2 or F3 | H | Ac | Boc |
| Ac | F2 or F3 | Ac | Boc |
| Ac | H | F2 or F3 | Boc |
| Ac | H | Ac | F2 or F3 |

The prior disclosure illustrates how one may modify prior art taxanes to make them magnetic. As will be apparent to those skilled in the art, one may similarly modify other anti-mitotic compounds to make them magnetic.

Other Modifiable Compounds

Many anti-mitotic compounds that may be modified in accordance with the process of this invention are described in the prior art, e.g., discodermolide, U.S. Pat. No. 6,541,509, hereby incorporated by reference.

Elsewhere in this specification, applicants teach how to make “magnetic taxanes” by incorporating therein various linker groups and/or siderophores. The same linker groups and/or siderophores may be utilized via substantially the same process to make discodermolide magnetic in the same manner.

As is disclosed elsewhere in this specification, siderophores are a class of compounds that act as chelating agents for various metals. When used to make “magnetic taxanes,” they are preferably bound to either the C7 and/or the C10 carbons of the paclitaxels. They can similarly be used to make “magnetic discodermolides,” but in this latter case they should be bonded at the C17 carbon of discodermolide, to which a hydroxyl group is bound. The same linker that is used to link the C7/C10 carbon of the taxane to the siderophores may also be used to link the C17 carbon of the discodermolide to the siderophore.

In one embodiment, the “siderophoric group” disclosed in U.S. Pat. No. 6,310,058, is used. The U.S. Pat. No. 6,310,058 is incorporated by reference. The siderophoric group is of the formula —(CH₂)ₘ—N(OH)—C(O)—(CH₂)ₙ—(CH═CH)ₒ—CH₃, wherein m is an integer of from 2 to 6, n is 0 or an integer of from 1 to 22, and o is 0 or an integer 1 to 4, provided that m+o is no greater than 25.

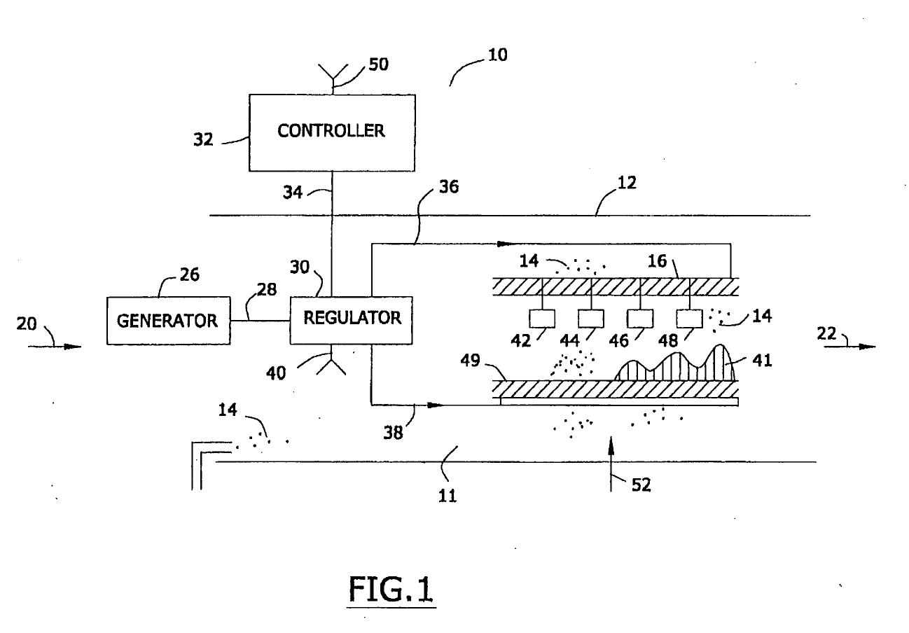

In another embodiment, “magnetic epothilone A” and/or “magnetic epothilones B” is made by a similar process. As is also disclosed in the FIG. 1 of the Kowalski et al. article (see page 614), and in the formula depicted, the epothilone A exists when, in such formula, the alkyl group (“R”) is hydrogen, whereas the epothilone B exists when, in such formula, the alkyl group is methyl. In either case, one can make magnetic analogs of these compounds by using the same siderophores and the same linkers groups but utilizing them at a different site. One may bind such siderophores at either the number 3 carbon (to which a hydroxyl group is bound) and/or the number 7 carbon (to which another hydroxyl group is bound.).

Without wishing to be bound to any particular theory, it may be that the binding of the siderophores at the specified carbon sites imparts the required magnetic properties to such modified materials without adversely affecting the anti-mitotic properties of the material. In some embodiments, the anti-mitotic properties of the modified magnetic materials surpass the anti-mitotic properties of the unmodified materials.

This is unexpected; for, if the same linker groups and/or siderophores are used to bind to other than the specified carbon atoms, materials with no or substantially poorer anti-mitotic properties are produced.

Referring to the magnetic taxanes described elsewhere in this specification (and also to FIG. 1 of the Kowalski et al. article), one should not link such siderophores to any carbons on the pendant aromatic rings. Referring to the discodermolide structure, one should not link siderophores to any of 1, 2, 3, or 4 carbon atoms. Referring to the epothilones, one should not link the siderophores to any carbon on the ring structure containing sulfur and nitrogen.

By way of further illustration, and referring to U.S. Pat. Nos. 5,504,074, 5,661,143, 5,892,069, 6,528,676, and 6,723,858 (the entire disclosure of each of which is hereby incorporated by reference into this specification), one may modify estradiol and estradiol metabolites to make them magnetic in accordance with the process of this invention.

Estradiol and estradiol metabolites such as 2-methoxyestradiol have been reported to inhibit cell division (See, e.g., U.S. Pat. No. 6,723,858; Seegers, J. C. et al. J. Steroid Biochem. 32, 797-809 (1989); Lottering, M-L. et al. Cancer Res. 52, 5926-5923 (1992); Spicer, L. J. and Hammond, J. M. Mol. and Cell. Endo. 64, 119-126 (1989); Rao, P. N. and Engelberg, J. Exp. Cell Res. 48, 71-81 (1967)).

In one embodiment, the modifiable anti-mitotic agent is an anti-microtubule agent. Representative anti-microtubule agents include, e.g., “ . . . taxanes (e.g., paclitaxel and docetaxel), camptothecin, eleutherobin, sarcodictyins, epothilones A and B, discodermolide, deuterium oxide (D₂O), hexylene glycol (2-methyl-2,4-pentanediol), tubercidin (7-deazaadenosine), LY290181 (2-amino-4-(3-pyridyl)-4H-naphtho(1,2-b)pyran-3-cardonitrile), aluminum fluoride, ethylene glycol bis-(succinimidylsuccinate), glycine ethyl ester, nocodazole, cytochalasin B, colchicine, colcemid, podophyllotoxin, benomyl, oryzalin, majusculamide C, demecolcine, methyl-2-benzimidazolecarbamate (MBC), LY195448, subtilisin, 1069C85, steganacin, combrestatin, curacin, estradiol, 2-methoxyestradiol, flavanol, rotenone, griseofulvin, vinca alkaloids, including vinblastine and vincristine, maytansinoids and ansamitocins, rhizoxin, phomopsin A, ustiloxins, dolastatin 10, dolastatin 15, halichondrins and halistatins, spongistatins, cryptophycins, rhazinilam, betaine, taurine, isethionate, HO-221, adociasulfate-2, estramustine, monoclonal anti-idiotypic antibodies, microtubule assembly promoting protein (taxol-like protein, TALP), cell swelling induced by hypotonic (190 mosmol/L) conditions, insulin (100 nmol/L) or glutamine (10 mmol/L), dynein binding, gibberelin, XCHO1 (kinesin-like protein), lysophosphatidic acid, lithium ion, plant cell wall components (e.g., poly-L-lysine and extensin), glycerol buffers, Triton X-100 microtubule stabilizing buffer, microtubule associated proteins (e.g., MAP2, MAP4, tau, big tau, ensconsin, elongation factor-1-alpha (EF-1.alpha.) and E-MAP-115), cellular entities (e.g., histone H1, myelin basic protein and kinetochores), endogenous microtubular structures (e.g., axonemal structures, plugs and GTP caps), stable tubule only polypeptide (e.g., STOP145 and STOP220) and tension from mitotic forces, as well as any analogues and derivatives of any of the above. Within other embodiments, the anti-microtubule agent is formulated to further comprise a polymer.”

The term “anti-microtubule,” refers to any “ . . . protein, peptide, chemical, or other molecule which impairs the function of microtubules, for example, through the prevention or stabilization of polymerization. A wide variety of methods may be utilized to determine the anti-microtubule activity of a particular compound, including for example, assays described by Smith et al. (Cancer Lett 79(2):213-219, 1994) and Mooberry et al., (Cancer Lett. 96(2):261-266, 1995);” see, e.g., lines 13-21 of column 14 of U.S. Pat. No. 6,689,803. One method, utilizing the anti-mitotic factor, is described in this specification.

An extensive listing of anti-microtubule agents is provided in columns 14, 15, 16, and 17 of U.S. Pat. No. 6,689,803; and one or more of them may be modified in accordance with the process of this invention to make them magnetic.

By way of yet further illustration, one may use one or more of the anti-mitotic agents disclosed in U.S. Pat. Nos. 6,673,937 (syntheses and methods of use of new antimitotic agents), 6,624,317 (taxoid conjugates as antimitotic and antitumor agents), 6,593,334 (camptothecin-taxoid conjugates as antimitotic and antitumor agents), 6,593,321 (2-alkoxyestradiiol analogs with antiproliferative and antimitotic activity), 6,569,870 (fluorinated quinolones as antimitotic and antitumor agent), 6,528,489 (mycotoxin derivatives as antimitotic agents), 6,392,055 (synthesis and biological evaluation of analogs of the antimitotic marine natural product curacin A), 6,127,377 (vinka alkaloid antimitotic halogenated derivatives), 5,695,950 (method of screening for antimitotic compounds using the cdc25 tyrosine phosphatase), 5,620,985 (antimitotic binary alkaloid derivatives from catharanthus roseus), 5,294,538 (method of screening for antimitotic compounds using the CDC tyrosine phosphatase), and the like. The entire disclosure of each of these United States patent Nos. is hereby incorporated by reference into this specification.

As will be apparent, one or more of the aforementioned anti-mitotic and/or anti-microtubule agents may be modified to make them magnetic in accordance with this invention.

Synergistic Combinations of Magnetic Anti-Mitotic Agents

In one embodiment of this invention, discussed elsewhere in this specification, a synergistic combination of the magnetic anti-mitotic compound of this invention and paclitaxel is described. In the embodiment of the invention described in this section of the specification, a synergistic combination of two or more anti-mitotic compounds is described.

In one embodiment, the first anti-mitotic compound is a magnetic taxane such as, e.g., magnetic paclitaxel and/or magnetic docetaxel. In this embodiment, the second anti-mitotic compound may be magnetic discodermolide, and/or magnetic epothilone A, and/or magnetic epothilone B, and/or mixtures thereof.

Gadolinium Complexes

In addition to the foregoing magnetic-antimitotic complexes, complexes of antimitotic compounds with gadolinium are also useful in the present invention. Various gadolinium compounds are useful as MRI contrast agents, and they can be exploited here to facilitate locating and imaging the tumor.

Gadolinium has a higher magnetic moment than iron, and a very special ground state (“S state”) that permits it to interact strongly with other gadolinium compounds and less so with non-gadolinium compounds. By conjugating a gadolinium-containing magnetic group with an anti-mitotic compound such as paclitaxel, strong magnetic interactions can be achieved between such groups and a magnetic force from an externally applied magnetic field. A static field focused on the tumor site would draw those compounds preferentially towards the tumor site; meanwhile, an RF oscillating magnetic field, such as is used in MRI, could simultaneously monitor the delivery of the drug as gadoliunium provides a very good signal-to-noice ratio as an fMRI agent.

Various gadolinium-containing magnetic moieties and gadolinium ligands are disclosed in the literature, as are methods for derivatizing molecules to add such moieties. One such suitable Gadolinium complex is Diethylenetriaminepentaacetate (DPTA), which has the added advantage of similarity in structure with deferroxamine, but having greater chelation ability. Additional suitable gadolinium complexes are disclosed in É. Girard et al, Biological Crystallography, 59, Part 1, 118-126, January 2003 (disclosing the gadolinium complexes: D03A, 1,4,7,10-tetraazacyclododecane-1,4,7-triacetic acid; DOTA, 1,4,7,10-tetraazacyclododecane-1,4,7,10-tetraacetic acid; HPDO3A, 10-(2-hydroxypropyl)-1,4,7,10-tetraazacyclododecane-1,4,7-triacetic acid; DOTMA, a,a′,a″,a′″-tetramethyl-1,4,7,10-tetraazacyclododecane-1,4,7,10-tetraacetic acid; HPSA-DO3A, 10-(2-{[2-hydroxy-1-(hydroxymethyl)ethyl]amino}-1-(hydroxymethyl)-2-oxoethyl)-1,4,7,10-tetraazacyclododecane-1,4,7-triacetic acid; DTPA-BMA, diethylenetriaminepentaacetic acid bismethylamide; DTPA, diethylenetriaminepentaacetic acid).

The exploitation of such gadolinium-containing magnetic moieties, ligands, and methods to create magnetic gadolinium-antimitotic complexes are within the competency of one of ordinary skill in the art without undue experimentation.

Properties of the Preferred Anti-Mitotic Compounds

In one preferred embodiment, the compound of this invention has a mitotic index factor of at least about 10 percent and, more preferably, at least about 20 percent. In one aspect of this embodiment, the mitotic index factor is at least about 30 percent. In another embodiment, the mitotic index factor is at least about 50 percent.

In another embodiment of the invention, the compound of this invention has a mitotic index factor of less than about 5 percent.

As is known to those skilled in the art, the mitotic index is a measure of the extent of mitosis. Reference may be had, e.g., to U.S. Pat. Nos. 5,262,409 (binary tumor therapy), 5,443,962 (methods of identifying inhibitors of cdc25 phosphatase), 5,744,300 (methods and reagents for the identification and regulation of senescence-related genes), 6,613,318, 6,251,585 (assay and reagents for identifying anti-proliferative agents), 6,252,058 (sequences for targeting metastatic cells), 6,387,642 (method for identifying a reagent that modulates Myt1 activity), U.S. Pat. No. 6,413,735 (method of screening for a modulator of angiogenesis), U.S. Pat. No. 6,531,479 (anti-cancer compounds), 6,599,694 (method of characterizing potential therapeutics by determining cell-cell interactions), 6,620,403 (in vivo chemo sensitivity screen for human tumors), 6,699,854 (anti-cancer compounds), 6,743,576 (database system for predictive cellular bioinformatics), and the like. The entire disclosure of each of these United States patent Nos. is hereby incorporated by reference into this specification.

By means of yet further illustration, one may measure the mitotic index by means of the procedures described in, e.g., articles by Keila Torres et al. (“Mechanisms of Taxol-Induced Cell Death are Concentration Dependent,” Cancer Research 58, 3620-3626, Aug. 15, 1998), and Jie-Gung Chen et al. (“Differential Mitosis Responses to Microtubule-stabilizing and destabilizing Drugs,” Cancer Research 62, 1935-1938, Apr. 1, 2002).

The mitotic index is preferably measured by using the well-known HeLa cell lines. As is known to those skilled in the art, HeLa cells are cells that have been derived from a human carcinoma of the cervix from a patient named Henrietta Lack; the cells have been maintained in tissue culture since 1953. The HeLa cell line can be obtained from the American Type Culture Collection, Manassas Va.

The compound of this invention preferably has a molecular weight of at least about 150 grams per mole. In one embodiment, the molecular weight of such compound is at least 300 grams per mole. In another embodiment, the molecular weight of such compound is 400 grams per mole. In yet another embodiment, the molecular weight of such compound is at least about 550 grams per mole. In yet another embodiment, the molecular weight of such compound is at least about 1,000 grams per mole. In yet another embodiment, the molecular weight of such compound is at least 1,200 grams per mole.

The compound of this invention preferably has a positive magnetic susceptibility of at least 1,000×10−6 centimeter-gram-seconds (cgs). As is known to those skilled in the art, magnetic susceptibility is the ratio of the magnetization of a material to the magnetic field strength. Reference may be had, e.g., to U.S. Pat. Nos. 3,614,618, 3,644,823, 3,657,636, 3,665,297, 3,758,847, 3,758,848, 3,879,658, 3,890,563, 3,980,076, 4,079,730, 4,277,750, 4,359,399, 4,507,613, 4,662,359, 4,701,712, 5,233,992, 6,208,884, 6,321,105, and the like. The entire disclosure of each of these U.S. patent applications is incorporated by reference.

In one embodiment, the compound of this invention has a positive magnetic susceptibility of at least 5,000×10−6 cgs. In another embodiment, such compound has a positive magnetic susceptibility of at least 10,000×10−6 cgs.

The compound of this invention is preferably comprised of at least 7 carbon atoms and, more preferably, at least about 10 carbon atoms. In another embodiment, such compound is comprised of at least 13 carbon atoms and at least one aromatic ring; in one aspect of this embodiment, the compound has at least two aromatic rings. In another embodiment, such compound is comprised of at least 17 carbon atoms.

In one embodiment, the compound of this invention is comprised of at least one oxetane ring. As is disclosed, e.g., on page 863 of N. Irving Sax's “Hawley's Condensed Chemical Dictionary,” Eleventh Edition (Van Nostrand Reinhold Company, New York, N.Y., 1987), the oxetane group, also known as “trimethylene oxide), is identified by chemical abstract number CAS: 503-30-0. The oxetane group present in the preferred compound preferably is unsubstituted. In one embodiment, however, one ore more of the ring carbon atoms (either carbon number one, or carbon number two, or carbon number 3), has one or more of its hydrogen atoms substituted by a halogen group (such as chlorine), a lower alkyl group of from 1 to 4 carbon atoms, a lower haloalkyl group of from 1 to 4 carbon atoms, a cyanide group (CN), a hydroxyl group, a carboxyl group, an amino group (which can be primary, secondary, or tertiary and may also contain from 0 to 6 carbon atoms), a substituted hydroxyl group (such as, e.g., an ether group containing from 1 to 6 carbon atoms), and the like. In one aspect of this embodiment, the substituted oxetane group is 3,3-bis (chloromethyl) oxetane.

In one embodiment, the compound of this invention is comprised of from about 1 to 10 groups of the formula —OB, in which B is selected from the group consisting of hydrogen, alkyl of from about 1 to about 5 carbon atoms, and a moiety of the formula R—(C=0)-O—, wherein R is selected from the group consisting of hydrogen and alkyl of from about 1 to about 6 carbon atoms, and the carbon is bonded to the R moiety, to the double-bonded oxygen, and to the single bonded oxygen, thereby forming what is commonly known as an acetyl group. This acetyl group preferably is linked to a ring structure that is unsaturated and preferably contains from about 6 to about 10 carbon atoms.

In one embodiment, the compound is comprised of two unsaturated ring structures linked by an amide structure, which typically has an acyl group, —CONR1-, wherein R1 is selected from the group consisting of hydrogen, lower alkyl of from 1 to about 6 carbon atoms. In one preferred embodiment, the N group is bonded to both to the R1 group and also to radical that contains at least about 20 carbon atoms and at least about 10 oxygen atoms.

In one embodiment, the compound of this invention contains at least one saturated ring comprising from about 6 to about 10 carbon atoms. By way of illustration, the saturated ring structures may be one or more cyclohexane rings, cycloheptane rings, cyclooctane rings, cylcononane rings, and/or cyclodecane rings. In one aspect of this embodiment, at least one saturated ring in the compound is bonded to at least one quinine group.

In one embodiment, the compound of this invention may comprise a ring structure with one double bond or two double bonds (as opposed to the three double bonds in the aromatic structures). These ring structures may be a partially unsaturated material selected from the group consisting of partially unsaturated cyclohexane, partially unsaturated cycloheptane, partially unsaturated cyclooctane, partially unsaturated cyclononane, partially unsaturated cyclodecane, and mixtures thereof.

The compound of this invention may also be comprised of at least one inorganic atom with a positive magnetic susceptibility of at least 200×10−6 cgs. Thus, and referring to the “CRC Handbook of Chemistry and Physics,” 63rd Edition (CRC Press, Inc., Boca Raton, Fla., 1982-83), the magnetic susceptibility of elements are described at pages E-118 to E-123. Suitable inorganic (i.e., non-carbon containing) elements with a positive magnetic susceptibility greater than about 200×10−6 cgs include, e.g., cerium (+5,160×10−6 cgs), cobalt (+11,000×10−6 cgs), dysprosium (+89,600×10−6 cgs), europium (+34,000×10−6 cgs), gadolinium (+755,000×10−6 cgs), iron (+13,600×10−6 cgs), manganese (+529×10−6 cgs), palladium (+567.4×10−6 cgs), plutonium (+610×10−6 cgs), praseodymium (+5010×10−6 cgs), samarium (+2230×10−6 cgs), technetium (+250×10−6 cgs), thulium (+51,444×10−6 cgs), and the like. In one embodiment, the positive magnetic susceptibility of such element is greater than about +500×10−6 cgs and, preferably, greater than about +1,000×10−6 cgs.

In one compound, the inorganic atom is radioactive. As is known to those skilled in the art, radioactivity is a phenomenon characterized by spontaneous disintegration of atomic nuclei with emission of corpuscular or electromagnetic radiation.

In another preferred embodiment, one or more inorganic or organic atoms that do not have the specified degree of magnetic susceptibility are radioactive. Thus, e.g., the radioactive atom may be, e.g., radioactive carbon, radioactive hydrogen (tritium), radioactive phosphorus, radioactive sulfur, radioactive potassium, or any other of the atoms that exist is radioactive isotope form.

One preferred class of atoms is the class of radioactive nuclides. As is known to those skilled in the art, radioactive nuclides are atoms that disintegrate by emission of corpuscular or electromagnetic radiation. The rays most commonly emitted are alpha or beta gamma rays. See, e.g., page F-109 of the “CRC Handbook of Chemistry and Physics.”

Radioactive nuclides are well known and are described, e.g., in U.S. Pat. Nos. 4,355,179 (radioactive nuclide labeled propiophenone compounds), 4,625,118 (device for the elution and metering of a radioactive nuclide), 5,672,876 (method and apparatus for measuring distribution of radioactive nuclide in a subject), and 6,607,710 (bisphosphonic acid derivative and compound thereof labeled with radioactive nuclide.). The entire disclosure of each of these United States patent Nos. is hereby incorporated by reference into this specification.

Suitable nuclides include, but are not limited to, cobalt 53, cobalt 54, cobalt 55, cobalt 56, cobalt 57, cobalt 58, cobalt 59, cobalt 60, cobalt 61, cobalt 62, cobalt 63, gadolinium 146, iron 49, iron 51, iron 52, iron 53, iron 54, iron 57, iron 58, iron 59, iron 60, iron 61, iron 62, manganese 50, praseodymium 135, and samarium 156.

The compound of this invention preferably has a magnetic moment of at least about 0.5 Bohr magnetrons per molecule and, more preferably, at least about 1.0 Bohr magnetrons per molecule. In one embodiment, the compound has a magnetic moment of at least about 2 Bohr magnetrons per molecule.

As is known to those skilled in the art, a Bohr magnetron is the amount he/4(pi)mc, wherein he is Plank's constant, e and m are the charge and mass of the electron, c is the speed of light, and pi is equal to about 3.14567. Reference may be had, e.g., to U.S. Pat. Nos. 4,687,331, 4,832,877, 4,849,107, 5,040,373 (“(One Bohr magnetron is equal to 9.273×10−24 Joules/Tesla”), 5,169,944, 5,323,227 (“μo is a constant known as the Bohr magnetron at 9.274×10−21 erg/Gauss”), 5,352,979 6,383,597, 6,725,668, 6,739,137 (“One Bohr magnetron μB is equal to 9.273×10−24 Joules/Tesla”), and the like. The entire disclosure of each of these United States patent Nos. is hereby incorporated by reference into this specification.

In one preferred embodiment, the magnetic compound of this invention is water soluble. As used in this specification, the term “water soluble” refers to a solubility of at least 10 micrograms per milliliter and, more preferably, at least 100 micrograms per milliliter. By way of comparison, the solubility of paclitaxel in water is only about 0.4 micrograms per milliliter.

In one embodiment, the magnetic compound of this invention has a water solubility of at least 500 micrograms per milliliter, and more preferably at least 1,000 micrograms per milliliter. In yet another embodiment, the magnetic compound of this invention has a water solubility of at least 2500 micrograms per milliliter. In yet another embodiment, the magnetic compound of this invention has a water solubility of at least 5,000 micrograms per milliliter. In yet another embodiment, the magnetic compound of this invention has a water solubility of at least 10,000 micrograms per milliliter.

In another embodiment, the magnetic compound of this invention has a water solubility of less than about 10 micrograms per milliliter and, preferably, less than about 1.0 micrograms per milliliter.

Without wishing to be bound to any particular theory, the presence of a hydrophilic group in the compound enhances water solubility. Thus, a siderophore group enhances water-solubility. As is known to those skilled in the art, a siderophore is one of a number of low molecular weight, iron-containing, or iron binding organic compounds or groups. Siderophores have a strong affinity for Fe3+ (which they chelate) and function in the solubilization and transport of iron. Siderophores are classified as belonging to either the phenol-catechol type (such as enterobactin and agrobactin), or the hydroxamic acid type (such as ferrichrome and mycobactin). J. Stenesh, “Dictionary of Biochemistry and Molecular Biology,” Second Edition, p. 442 (John Wiley & Sons, New York, N.Y., 1989).

In one embodiment, the compound of this invention is comprised of one or more siderophore groups bound to a magnetic moiety (such as, e.g., an atom selected from the group consisting of iron, cobalt, nickel, and mixtures thereof).

The inclusion of other hydrophilic groups is contemplated. Thus, in place of or in addition to such siderophore group, one might use hydrophilic groups such as hydroxyl groups, carboxyl groups, amino groups, organometallic ionic structures, phosphate groups, and the like. In one aspect, the hydrophilic group is biologically inert.

In one embodiment, the magnetic compound of this invention has an association rate with microtubules of at least 3,500,000/mole/second. The association rate may be determined in accordance with J. F. Diaz et al., “Fast Kinetics of Taxol Binding to Microtubules,” Journal of Biological Chemistry, 278(10) 8407-8455; see also, J. R. Strobe et al., Journal of Biological Chemistry, 275: 26265-26276 (2000).

In another embodiment of the invention, the magnetic compound of this invention has a dissociation rate with microtubules less than about 0.08/second, when measured at a temperature of 37 degrees Celsius and under atmospheric conditions. Thus, in this embodiment, the magnetic compound of this invention binds more durably to microtubules than does paclitaxel, which has a dissociation rate of at least 0.91/second.

In one embodiment, the dissociation rate of the magnetic compound of this invention is less than 0.7/second and, more preferably, less than 0.6/second.

In one embodiment of this invention, the anti-mitotic compound of the invention has the specified degree of water-solubility and of anti-mitotic activity but does not necessarily possess one or more of the magnetic properties described hereinabove.

Other Magnetic Compounds

In another embodiment of this invention, other compounds which are not necessarily anti-mitotic are made magnetic by a process comparable to the process described in this specification for making taxanes magnetic.

In this embodiment, it is preferred to make “magnetic derivatives” of drugs and therapeutic agents. These derivative compounds each preferably have a molecular weight of at least 150 grams per mole, a positive magnetic susceptibility of at least 1,000×10−6 cgs, and a magnetic moment of at least 0.5 Bohr magnetrons, wherein said compound is comprised of at least 7 carbon atoms and at least one inorganic atom with a positive magnetic susceptibility of at least 200×10−6 cgs.

Some of the preferred “precursors” used to make these “derivative compounds” are described in the remainder of this section of the specification.

The precursor materials may be either proteinaceous or non-proteinaceous drugs, as those terms are defined in U.S. Pat. No. 5,194,581, the entire disclosure of which is hereby incorporated by reference into this specification.

U.S. Pat. No. 5,194,581 discloses “The drugs with which can be incorporated in the compositions of the invention include non-proteinaceous as well as proteinaceous drugs. The term “non-proteinaceous drugs” encompasses compounds which are classically referred to as drugs such as, for example, mitomycin C, daunorubicin, vinblastine, AZT, and hormones. Similar substances are within the skill of the art. The proteinaceous drugs which can be incorporated in the compositions of the invention include immunomodulators and other biological response modifiers. The term “biological response modifiers” is meant to encompass substances which are involved in modifying the immune response in such manner as to enhance the particular desired therapeutic effect, for example, the destruction of the tumor cells. Examples of immune response modifiers include such compounds as lymphokines. Examples of lymphokines include tumor necrosis factor, the interleukins, lymphotoxin, macrophage activating factor, migration inhibition factor, colony stimulating factor and the interferons. Interferons which can be incorporated into the compositions of the invention include alpha-interferon, beta-interferon, and gamma-interferon and their subtypes. In addition, peptide or polysaccharide fragments derived from these proteinaceous drugs, or independently, can also be incorporated. Also, encompassed by the term “biological response modifiers” are substances generally referred to as vaccines wherein a foreign substance, usually a pathogenic organism or some fraction thereof, is used to modify the host immune response with respect to the pathogen to which the vaccine relates. Those of skill in the art will know, or can readily ascertain, other substances which can act as proteinaceous drugs.”

The precursor may be a lectin, as is disclosed in U.S. Pat. No. 5,176,907, the entire disclosure of which is hereby incorporated by reference into this specification. This United States patent No. discloses “Lectins are proteins, usually isolated from plant material, which bind to specific sugar moieties. Many lectins are also able to agglutinate cells and stimulate lymphocytes. Other therapeutic agents which can be used therapeutically with the biodegradable compositions of the invention are known, or can be easily ascertained, by those of ordinary skill in the art.”

In one embodiment, and referring to U.S. Pat. No. 5,420,105 (the entire disclosure of which is hereby incorporated by reference into this specification), the precursor material is selected from the group consisting of an anti-cancer anthracycline antibiotic, cis-platinum, methotrexate, vinblastine, mitoxanthrone ARA-C, 6-mercaptopurine, 6-mercaptoguanosine, mytomycin C and a steroid.

By way of further illustration, the precursor material is selected from the group consisting of antithrombogenic agents, antiplatelet agents, prostaglandins, thrombolytic drugs, antiproliferative drugs, antirejection drugs, antimicrobial drugs, growth factors, and anticalcifying agents.

By way of yet further illustration, the precursor material may, e.g., be any one or more of the therapeutic agents disclosed in column 5 of U.S. Pat. No. 5,464,650. The precursor material may be one or more of the drugs disclosed in U.S. Pat. No. 5,599,352, and WO 91/12779, the entire disclosures of which are hereby incorporated by reference.

By way of yet further illustration, the precursor may be any of the selected therapeutic drugs disclosed in U.S. Pat. Nos. 5,605,696 and 5,700,286 (the disclosures of which are hereby incorporated by reference into this specification).

By way of yet further illustration, and referring to U.S. Pat. No. 5,900,433 (the entire disclosure of which is hereby incorporated by reference into this specification), the precursor material may be a congener of an endothelium-derived bioactive composition of matter.

By way of yet further illustration, the precursor material may be heparin. See, U.S. Pat. No. 6,120,536 (the entire disclosure of which is hereby incorporated by reference into this specification). Alternatives to heparin include: antithrombolytics, anticoagulants, antibiotics, antiplatelet agents, thrombolytics, antiproliferatives, steroidal and non-steroidal antinflammatories, agents that inhibit hyperplasia and in particular restenosis, smooth muscle cell inhibitors, growth factors, growth factor inhibitors, cell adhesion inhibitors, cell adhesion promoters and drugs that may enhance the formation of healthy neointimal tissue, including endothelial cell regeneration.

By way of yet further illustration, and referring to U.S. Pat. No. 6,624,138 (the entire disclosure of which is hereby incorporated by reference into this specification), the precursor material may be one or more of the drugs described in this patent. Drugs contemplated for use in the compositions include the following categories and examples of drugs and alternative forms of these drugs such as alternative salt forms, free acid forms, free base forms, and hydrates: analgesics/antipyretics. (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate); antiasthamatics (e.g., ketotifen and traxanox); antibiotics (e.g., neomycin, streptomycin, chloramphenicol, cephalosporin, ampicillin, penicillin, tetracycline, and ciprofloxacin); antidepressants (e.g., nefopam, oxypertine, doxepin, amoxapine, trazodone, amitriptyline, maprotiline, phenelzine, desipramine, nortriptyline, tranylcypromine, fluoxetine, doxepin, imipramine, imipramine pamoate, isocarboxazid, trimipramine, and protriptyline); antidiabetics (e.g., biguanides and sulfonylurea derivatives); antifungal agents (e.g., griseofulvin, ketoconazole, itraconizole, amphotericin B, nystatin, and candicidin); antihypertensive agents (e.g., propanolol, propafenone, oxyprenolol, nifedipine, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine); anti-inflammatories (e.g., (non-steroidal) indomethacin, ketoprofen, flurbiprofen, naproxen, ibuprofen, ramifenazone, piroxicam, (steroidal) cortisone, dexamethasone, fluazacort, celecoxib, rofecoxib, hydrocortisone, prednisolone, and prednisone); antineoplastics (e.g., cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, and piposulfan); antianxiety agents (e.g., lorazepam, buspirone, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene); immunosuppressive agents (e.g., cyclosporine, azathioprine, mizoribine, and FK506 (tacrolimus)); antimigraine agents (e.g., ergotamine, propanolol, isometheptene mucate, and dichloralphenazone); sedatives/hypnotics (e.g., barbiturates such as pentobarbital, pentobarbital, and secobarbital; and benzodiazapines such as flurazepam hydrochloride, triazolam, and midazolam); antianginal agents (e.g., beta-adrenergic blockers; calcium channel blockers such as nifedipine, and diltiazem; and nitrates such as nitroglycerin, isosorbide dinitrate, pentearythritol tetranitrate, and erythrityl tetranitrate); antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, and prochlorperazine); antimanic agents (e.g., lithium carbonate); antiarrhythmics (e.g., bretylium tosylate, esmolol, verapamil, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine); antiarthritic agents (e.g., phenylbutazone, sulindac, penicillanine, salsalate, piroxicam, azathioprine, indomethacin, meclofenamate, gold sodium thiomalate, ketoprofen, auranofin, aurothioglucose, and tolmetin sodium); antigout agents (e.g., colchicine, and allopurinol); anticoagulants (e.g., heparin, heparin sodium, and warfarin sodium); thrombolytic agents (e.g., urokinase, streptokinase, and alteplase); antifibrinolytic agents (e.g., aminocaproic acid); hemorheologic agents (e.g., pentoxifylline); antiplatelet agents (e.g., aspirin); anticonvulsants (e.g., valproic acid, divalproex sodium, phenyloin, phenyloin sodium, clonazepam, primidone, phenobarbitol, carbamazepine, amobarbital sodium, methsuximide, metharbital, mephobarbital, mephenyloin, phensuximide, paramethadione, ethotoin, phenacemide, secobarbitol sodium, clorazepate dipotassium, and trimethadione); antiparkinson agents (e.g., ethosuximide); antihistamines/antipruritics (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, methdilazine; agents useful for calcium regulation (e.g., calcitonin, and parathyroid hormone); antibacterial agents (e.g., amikacin sulfate, aztreonam, chloramphenicol, chloramphenicol palirtate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate); antiviral agents (e.g., interferon alpha, beta or gamma, zidovudine, amantadine hydrochloride, ribavirin, and acyclovir); antimicrobials (e.g., cephalosporins such as cefazolin sodium, cephradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, clarithromycin); anti-infectives (e.g., GM-CSF); bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; inhalant corticosteroids such as beclomethasone dipropionate (BDP), and beclomethasone dipropionate monohydrate; salbutamol; ipratropium bromide; budesonide; ketotifen; salmeterol; xinafoate; terbutaline sulfate; triamcinolone; theophylline; nedocromil sodium; metaproterenol sulfate; albuterol; flunisolide; fluticasone proprionate; steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium); hypoglycemic agents (e.g., human insulin, purified beef insulin, purified pork insulin, glyburide, chlorpropamide, glipizide, tolbutamide, and tolazamide); hypolipidemic agents (e.g., clofibrate, dextrothyroxine sodium, probucol, pravastitin, atorvastatin, lovastatin, and niacin); proteins (e.g., DNase, alginase, superoxide dismutase, and lipase); nucleic acids (e.g., sense or anti-sense nucleic acids encoding any therapeutically useful protein, including any of the proteins described herein); agents useful for erythropoiesis stimulation (e.g., erythropoietin); antiulcer/antireflux agents (e.g., famotidine, cimetidine, and ranitidine hydrochloride); antinauseants/antiemetics (e.g., meclizine hydrochloride, nabilone, prochlorperazine, dimenhydrinate, promethazine hydrochloride, thiethylperazine, and scopolamine); as well as other drugs useful in the compositions and methods described herein include mitotane, halonitrosoureas, anthrocyclines, ellipticine, ceftriaxone, ketoconazole, ceftazidime, oxaprozin, albuterol, valacyclovir, urofollitropin, famciclovir, flutamide, enalapril, mefformin, itraconazole, buspirone, gabapentin, fosinopril, tramadol, acarbose, lorazepan, follitropin, glipizide, omeprazole, fluoxetine, lisinopril, tramsdol, levofloxacin, zafirlukast, interferon, growth hormone, interleukin, erythropoietin, granulocyte stimulating factor, nizatidine, bupropion, perindopril, erbumine, adenosine, alendronate, alprostadil, benazepril, betaxolol, bleomycin sulfate, dexfenfluramine, diltiazem, fentanyl, flecainid, gemcitabine, glatiramer acetate, granisetron, lamivudine, mangafodipir trisodium, mesalamine, metoprolol fumarate, metronidazole, miglitol, moexipril, monteleukast, octreotide acetate, olopatadine, paricalcitol, somatropin, sumatriptan succinate, tacrine, verapamil, nabumetone, trovafloxacin, dolasetron, zidovudine, finasteride, tobramycin, isradipine, tolcapone, enoxaparin, fluconazole, lansoprazole, terbinafine, pamidronate, didanosine, diclofenac, cisapride, venlafaxine, troglitazone, fluvastatin, losartan, imiglucerase, donepezil, olanzapine, valsartan, fexofenadine, calcitonin, and ipratropium bromide. These drugs are generally considered to be water soluble. Any of these water-soluble drugs may be used as precursors in the process of this invention to make a composition with the desired magnetic properties.

Other drugs useful in the present invention include albuterol, adapalene, doxazosin mesylate, mometasone furoate, ursodiol, amphotericin, enalapril maleate, felodipine, nefazodone hydrochloride, valrubicin, albendazole, conjugated estrogens, medroxyprogesterone acetate, nicardipine hydrochloride, zolpidem tartrate, amlodipine besylate, ethinyl estradiol, omeprazole, rubitecan, amlodipine besylate/benazepril hydrochloride, etodolac, paroxetine hydrochloride, paclitaxel, atovaquone, felodipine, podofilox, paricalcitol, betamethasone dipropionate, fentanyl, pramipexole dihydrochloride, Vitamin D3 and related analogues, finasteride, quetiapine fumarate, alprostadil, candesartan, cilexetil, fluconazole, ritonavir, busulfan, carbamazepine, flumazenil, risperidone, carbemazepine, carbidopa, levodopa, ganciclovir, saquinavir, amprenavir, carboplatin, glyburide, sertraline hydrochloride, rofecoxib carvedilol, halobetasolproprionate, sildenafil citrate, celecoxib, chlorthalidone, imiquimod, simvastatin, citalopram, ciprofloxacin, irinotecan hydrochloride, sparfloxacin, efavirenz, cisapride monohydrate, lansoprazole, tamsulosin hydrochloride, mofafinil, clarithromycin, letrozole, terbinafine hydrochloride, rosiglitazone maleate, diclofenac sodium, lomefloxacin hydrochloride, tirofiban hydrochloride, telmisartan, diazapam, loratadine, toremifene citrate, thalidomide, dinoprostone, mefloquine hydrochloride, trandolapril, docetaxel, mitoxantrone hydrochloride, tretinoin, etodolac, triamcinolone acetate, estradiol, ursodiol, nelfinavir mesylate, indinavir, beclomethasone dipropionate, oxaprozin, flutamide, famotidine, nifedipine, prednisone, cefuroxime, lorazepam, digoxin, lovastatin, griseofulvin, naproxen, ibuprofen, isotretinoin, tamoxifen citrate, nimodipine, amiodarone, and alprazolam. Specific non-limiting examples of some drugs that fall under the above categories include paclitaxel, docetaxel and derivatives, epothilones, nitric oxide release agents, heparin, aspirin, coumadin, PPACK, hirudin, polypeptide from angiostatin and endostatin, methotrexate, 5-fluorouracil, estradiol, P-selectin Glycoprotein ligand-1 chimera, abciximab, exochelin, eleutherobin and sarcodictyin, fludarabine, sirolimus, tranilast, VEGF, transforming growth factor (TGF)-beta, Insulin-like growth factor (IGF), platelet derived growth factor (PDGF), fibroblast growth factor (FGF), RGD peptide, beta or gamma ray emitter (radioactive) agents, and dexamethasone, tacrolimus, actinomycin-D, batimastat etc.” These drugs also may be used in the process of this invention to make magnetic compositons. Another preferred compound of the invention

In another embodiment of this invention, there is provided a compound that, in spite of having a molecular weight in excess of 550, still has a water solubility in excess of about 10 micrograms per milliliter. In particular, there is provided a compound with a molecular weight of at least about 550, a water solubility of at least about 10 micrograms per milliliter, a pKa dissociation constant of from about 1 to about 15, and a partition coefficient of from about 1.0 to about 50.

The compound of this embodiment of the invention has a molecular weight of at least about 550. In one embodiment, this compound has a molecular weight of at least about 700.

The water solubility of this compound is at least about 1 micrograms per milliliter and, more preferably, at least about 10 micrograms per milliliter. In one embodiment, such compound has a water solubility of at least about 100 micrograms per milliliter. In yet another embodiment, such compound has a water solubility of at least about 1,000 micrograms per milliliter.

The compound of this embodiment of the invention has a pKa dissociation constant of from about 1 to about 15. The compound of this embodiment of the invention preferably has a partition coefficient of from about 1.0 to about 50.

In one embodiment, the compound of this invention has a tumor uptake of at least about 10 percent and, more preferably, at least about 20 percent. In one embodiment, the tumor uptake is at least about 30 percent. In yet another embodiment, the tumor uptake is at least about 50 percent. In yet another embodiment, the tumor uptake is at least about 70 percent.

Tumor uptake is the extent to which the compound is selectively taken up by tumors from blood. It may be determined by dissolving 1 milligram of the compound to be tested in 1 milliliter of “Cremophor EL,” a 1:1 (volume/volume) mixture of anhydrous ethanol and polyethoxylated castor oil. For a discussion of such “Cremophor EL,” reference may be had, e.g., to U.S. Pat. Nos. 5,591,715 (methods and compositions for reducing multidrug resistance), 5,686,488 (polyethoxylated castor oil products as anti-inflammatory agents), 5,776,891 (compositions for reducing multidrug resistance), and the like. The entire disclosure of each of these United States patent Nos. is incorporated by reference.

In one embodiment, the magnetic properties of the anti-mitotic compound of this invention are used in order to preferentially deliver such compound to a specified site. In another embodiment, the magnetic properties of the compounds and compositions of this invention which are not necessarily anti-mitotic but have the desired magnetic properties also may be used to deliver such compounds and/or compositions to a desired site.

Thus, by way of illustration, one may guide delivery of the compound of this invention with conventional magnetic focusing means. In one aspect of this embodiment, a magnetic field of a specified strength is focused onto a desired therapeutic site, such as a tumor to be treated, whereby the compound is selectively drawn to the therapeutic site and binds with tubulin molecules at the site. In one embodiment, the focused magnetic field has a field strength of at least about 6 Tesla in order to cause microtubules to move linearly. The magnetic field may, e.g., be focused for a period of at least about 30 minutes following the administration of the compound of this invention.

One may use any of the conventional magnetic field generators known to those skilled in the art to produce such a magnetic field. Thus, e.g., one may use one or more of the magnetic field generators disclosed in U.S. Pat. Nos. 6,503,364, 6,377,149; 6,353,375; 6,340,888; 6,336,989; 6,335,617; 6,313,632; 6,297,634; 6,275,128; 6,246,066; 6,114,929; 6,099,459; 5,795,212; 6,106,380; 5,839,944; 5,971,835; 5,951,369; 6,506,102; 6,267,651; 6,309,285; 5,929,732; and 6,488,615. The entire disclosure of each of these United States patent Nos. is hereby incorporated by reference into this specification.

The Use of Externally Applied Energy to Affect an Implanted Medical Device

The prior art discloses many devices in which an externally applied electromagnetic field (i.e., a field originating outside of a biological organism, such as a human body) is generated in order to influence one or more implantable devices disposed within the biological organism; these may be used in conjunction with anti-mitotic compound of this invention. Some examples of these devices are described in the following references, all of which are incorporated by reference: U.S. Pat. Nos. 3,337,776; 3,890,953; 3,890,953; 4,095,588; 4,323,075; 4,340,038; 4,361,153; 4,408,607; 4,416,283; 4,871,351; 3,731,861; 3,692,027; 3,923,060; 4,003,379; 3,951,147; 4,193,397; 4,221,219; 4,258,711; 4,077,405; 4,282,872; 4,270,532; 4,360,019 and 4,373,527.

U.S. Pat. No. 5,487,760 discloses an implantable signal transceiver disposed in an artificial heart valve; this transceiver may be used in the process of this invention in accordance with the aforementioned telemetry device; and the entire disclosure of this United States patent No. is hereby incorporated by reference into this specification. As will be apparent to those skilled in the art, the sensor/transceiver combination may advantageously be used in conjunction with the anti-mitotic compound of this invention, and/or microtubules.

U.S. Pat. No. 5,702,430 discloses an implantable power supply; the entire disclosure of such patent is hereby incorporated by reference into this specification. This implantable power supply may be used to supply power to either the compound of this invention, the treatment site, and/or one or more other devices from which a specified energy output is desired.

Columns 1 through 5 of U.S. Pat. No. 5,702,430 describes implantable pump assemblies that may be used, e.g., to deliver the anti-mitotic compound of this invention.

U.S. Pat. No. 3,842,440 to Karlson (incorporated herein by reference), discloses an implantable linear motor prosthetic heart and control system containing a pump having a piston-like member which is reciprocal within a magnetic field. The piston-like member includes a compressible chamber in the prosthetic heart which communicates with the vein or aorta.

U.S. Pat. Nos. 3,911,897 and 3,911,898 to Leachman, Jr. (incorporated herein by reference) disclose heart assist devices controlled in the normal mode of operation to copulsate and counterpulsate with the heart, respectively, and produce a blood flow waveform corresponding to the blood flow waveform of the heart being assisted. The heart assist device is a pump connected serially between the discharge of a heart ventricle and the vascular system.

U.S. Pat. No. 4,102,610 to Taboada et al. discloses a magnetically operated constant volume reciprocating pump which can be used as a surgically implantable heart pump or assist.

U.S. Pat. Nos. 4,210,409 and 4,375,941 to Child disclose a pump used to assist pumping action of the heart having a piston movable in a cylindrical casing in response to magnetic forces.

U.S. Pat. No. 4,965,864 to Roth discloses a linear motor using multiple coils and a reciprocating element containing permanent magnets which is driven by microprocessor-controlled power semiconductors. A plurality of permanent magnets is mounted on the reciprocating member.

U.S. Pat. No. 4,541,787 (incorporated herein by reference), describes a pump configuration wherein a piston containing a permanent magnet is driven in a reciprocating fashion along the length of a cylinder by energizing a sequence of coils positioned around the outside of the cylinder.

U.S. Pat. No. 4,610,658 to Buchwald et al. (incorporated herein by reference), discloses an implantable fluid displacement peritoneovenous shunt system. The system comprises a magnetically driven pump having a spool piston fitted with a disc flap valve.

U.S. Pat. No. 5,089,017 to Young et al. (incorporated herein by reference), discloses a drive system for artificial hearts and left ventricular assist devices comprising one or more implantable pumps driven by external electromagnets. The pump utilizes working fluid, such as sulfur hexafluoride to apply pneumatic pressure to increase blood pressure and flow rate.

U.S. Pat. No. 5,743,854 (incorporated herein by reference), discloses a device for inducing and localizing epileptiform activity that is comprised of a direct current (DC) magnetic field generator, a DC power source, and sensors adapted to be coupled to a patient's head; this direct current magnetic field generator may be used in conjunction with the anti-mitotic compound of this invention and/or an auxiliary device and/or tubulin and/or microtubules.

U.S. Pat. No. 5,803,897 (incorporated herein by reference), discloses a penile prosthesis system comprised of an implantable pressurized chamber, a reservoir, a rotary pump, a magnetically responsive rotor, and a rotary magnetic field generator. Such fluid pumping means may be used to facilitate the delivery of the anti-mitotic compound of this invention.

U.S. Pat. No. 5,810,015 (incorporated herein by reference), describes an implantable power supply that can convert non-electrical energy (such as mechanical, chemical, thermal, or nuclear energy) into electrical energy; the entire disclosure of this United States patent No. is hereby incorporated by reference into this specification. This power supply may be used to supply energy to the anti-mitotic compound of this invention and/or to tubulin and/or to microtubules.

Transcutaneous inductive recharging of batteries in implanted devices is disclosed for example in U.S. Pat. Nos. 3,923,060; 4,082,097; 4,143,661; 4,665,896; 5,279,292; 5,314,453; 5,372,605, and many others. See, e.g., U.S. Pat. Nos. 4,432,363 (use of light or heat to power a solar battery within an implanted device); 4,661,107 (discloses recharging of a pacemaker battery using mechanical energy created by motion of an implanted heart valve.) These may also be used in the present invention.

A number of implanted devices have been powered without batteries. U.S. Pat. Nos. 3,486,506 and 3,554,199 disclose generation of electric pulses in an implanted device by movement of a rotor in response to the patient's heartbeat; 3,563,245, discloses a miniaturized power supply unit which employs mechanical energy of heart muscle contractions to generate electrical energy for a pacemaker; 3,456,134, discloses a piezoelectric converter for electronic implants in which a piezoelectric crystal is in the form of a weighted cantilever beam capable of responding to body movement to generate electric pulses; 3,659,615, discloses a piezoelectric converter which reacts to muscular movement in the area of implantation; 4,453,537, discloses a pressure actuated artificial heart powered by a second implanted device attached to a body muscle which in turn is stimulated by an electric signal generated by a pacemaker. These can also be used in the present invention.

U.S. Pat. No. 5,945,762, the entire disclosure of which is hereby incorporated by reference into this specification, discloses an external transmitter adapted to magnetically excite an implanted receiver coil; such an implanted receiver coil may be disposed near, e.g., the anti-mitotic compound of this invention and/or other devices and/or tubulin and/or microtubules.

U.S. Pat. No. 5,954,758, the entire disclosure of which is hereby incorporated by reference into this specification, claims an implantable electrical stimulator comprised of an implantable radio frequency receiving coil, an implantable power supply, an implantable input signal generator, an implantable decoder, and an implantable electrical stimulator.

U.S. Pat. No. 6,006,133, the entire disclosure of which is hereby incorporated by reference into this specification, describes an implantable medical device comprised of a hermetically sealed housing. Such a hermetically sealed housing may be used to contain, e.g., the anti-mitotic compound of this invention.

U.S. Pat. No. 6,083,166, the entire disclosure of which is hereby incorporated by reference into this specification, discloses an ultrasound transmitter for use with a surgical device. This ultrasound transmitter may be used, e.g., to affect the anti-mitotic compound of this invention and/or tubulin and/or microtubules.

U.S. Pat. No. 6,152,882, the entire disclosure of which is hereby incorporated by reference into this specification, discloses an implantable electroporation unit, an implantable probe electrode, an implantable reference electrode, and an amplifier unit; this electroporation unit may be used to treat, e.g., cancer cells in conjunction with the anti-mitotic compound of this invention.

U.S. Pat. No. 6,169,925, the entire disclosure of which is hereby incorporated by reference into this specification, describes a transceiver for use in communication with an implantable medical device.

U.S. Pat. No. 6,185,452, the entire disclosure of which is hereby incorporated by reference into this specification, claims a device for stimulating internal tissue, wherein such device is comprised of: a sealed elongate housing configured for implantation in said patient's body, said housing having an axial dimension of less than 60 mm and a lateral dimension of less than 6 mm; power consuming circuitry carried by said housing including at least one electrode extending externally of said housing, said power consuming circuitry including a capacitor and pulse control circuitry for controlling (1) the charging of said capacitor and (2) the discharging of said capacitor to produce a current pulse through said electrode; a battery disposed in said housing electrically connected to said power consuming circuitry for powering said pulse control circuitry and charging said capacitor, said battery having a capacity of at least one microwatt-hour; an internal coil and a charging circuit disposed in said housing for supplying a charging current to said battery; an external coil adapted to be mounted outside of said patient's body; and means for energizing said external coil to generate an alternating magnetic field for supplying energy to said charging circuit via said internal coil. Such capacitative discharge energy may be used to affect either the anti-mitotic compound of this invention and/or tubulin and/or microtubules.

U.S. Pat. No. 6,235,024, the entire disclosure of which is hereby incorporated by reference into this specification, discloses an implantable high frequency energy generator; such high-frequency energy may be used to affect either the anti-mitotic compound of this invention, tubulin, microtubules, and/or one or more other implanted devices.

An implantable light-generating apparatus is described U.S. Pat. No. 6,363,279, the entire disclosure of which is hereby incorporated by reference into this specification. In one embodiment, the compound of this invention is comprised of a photolytic linker which is caused to disassociate upon being exposed to specified light energy.

An implantable ultrasound probe is described in claim 1 of U.S. Pat. No. 6,421,565, the entire disclosure of which is hereby incorporated by reference into this specification. Such ultrasound may be used, e.g., to treat the microtubules of cancer cells; and this treatment may be combined, e.g., with the anti-mitotic compounds of this invention.

An implantable stent that contains a tube and several optical emitters located on the inner surface of the tube is disclosed in U.S. Pat. No. 6,488,704, the entire disclosure of which is hereby incorporated by reference into this specification. One may use one or more of the implantable devices described in U.S. Pat. No. 6,488,704 together with the anti-mitotic compound of this invention and/or tubulin and/or microtubules and/or another in vivo device.

Many other implantable devices and configurations are described in U.S. Pat. No. 6,488,704. These devices and configurations may be used in conjunction with the anti-mitotic compound of this invention, and/or tubulin, and/or microtubules, and/or other auxiliary, implanted device.

The implantable stent of U.S. Pat. No. 6,488,704 may be comprised of implantable laser devices. These devices may advantageously be used in the process of this invention.

U.S. Pat. No. 6,585,763, the entire disclosure of which is hereby incorporated by reference into this specification, describes a vascular graft that may be used in the present invention.

U.S. Pat. No. 6,605,089, the entire disclosure of which is hereby incorporated by reference into this specification, discloses an implantable bone growth promoting device, which may be used in this invention.

U.S. Pat. No. 6,641,520, the entire disclosure of which is hereby incorporated by reference into this specification, discloses a magnetic field generator for providing a static or direct current magnetic field generator. The magnetic field generator described in this patent may be used in conjunction the anti-mitotic compound and/or tubulin and/or microtubules. In column 1 of this patent, some “prior art” magnetic field generators were described; and they also may be so used.

U.S. Pat. No. 6,663,555, the entire disclosure of which is incorporated by reference into this specification, also claims a magnetic field generator; this magnetic field generator may be used in conjunction with the anti-mitotic compound of this invention and/or tubulin and/or microtubules.

Published U.S. Patent application no. US2002/0182738 discloses an implantable flow cytometer. The entire disclosure of this published United States patent application no. is hereby incorporated by reference into this specification.

A similar flow cytometer is disclosed in published U.S. Patent application no. US2003/0036718, the entire disclosure of which is also hereby incorporated by reference into this specification.

The anti-mitotic compound of this invention may be used in conjunction with prior art polymeric carriers and/or delivery systems comprised of polymeric material. In one embodiment, the polymeric material is preferably comprised of one or more anti-mitotic compounds that are adapted to be released from the polymeric material when the polymeric material is disposed within a biological organism. The polymeric material may be, e.g., any of the drug eluting polymers known to those skilled in the art.

By way of illustration, and referring to U.S. Pat. No. 3,279,996 (the entire disclosure of which is hereby incorporated by reference into this specification), the polymeric material may be silicone rubber. One may use, as the anti-mitotic compound a material that is soluble in and capable of diffusing through the polymeric material.

At column 1 of U.S. Pat. No. 3,279,996, other “carrier agents” which may be used as polymeric material are also disclosed, including “ . . . beeswax, peanut oil, stearates, etc.” Any of these “carrier agents” may be used as the polymeric material.

By way of further illustration, and as is disclosed in U.S. Pat. No. 4,191,741 (the entire disclosure of which is hereby incorporated by reference into this specification), one may use dimethylpolysiloxane rubber as the polymeric material.

In column 1 of U.S. Pat. No. 4,191,741, other materials which may be used as the polymeric material are disclosed. Thus, it is stated in such patent that “Long et al. U.S. Pat. No. 3,279,996 describes an implant for releasing a drug in the tissues of a living organism comprising the drug enclosed in a capsule formed of silicone rubber. The drug migrates through the silicone rubber wall and is slowly released into the living tissues. A number of biocompatible silicone rubbers are described in the Long et al. patent. When a drug delivery system such as that described in U.S. Pat. No. 3,279,996 is used in an effort to administer estradiol to a ruminant animal a number of problems are encountered. For example, an excess of the drug is generally required in the hollow cavity of the implant. Also, it is difficult to achieve a constant rate of administration of the drug over a long time period such as from 200 to 400 days as would be necessary for the daily administration of estradiol to a growing beef animal. Katz et al. U.S. Pat. No. 4,096,239 describes an implant pellet containing estradiol or estradiol benzoate which has an inert spherical core and a uniform coating comprising a carrier and the drug. The coating containing the drug must be both biocompatible and biosoluble, i.e., the coating must dissolve in the body fluids which act upon the pellet when it is implanted in the body. The rate at which the coating dissolves determines the rate at which the drug is released. Representative carriers for use in the coating material include cholesterol, solid polyethylene glycols, high molecular weight fatty acids and alcohols, biosoluble waxes, cellulose derivatives and solid polyvinyl pyrrolidone.” The polymeric material used with the anti-mitotic compound is, in one embodiment, both biocompatible and biosoluble.

By way of yet further illustration, and referring to U.S. Pat. No. 4,429,080 (the entire disclosure of which is hereby incorporated by reference into this specification), the polymeric material may be a synthetic absorbable copolymer formed by copolymerizing glycolide with trimethylene carbonate.

By way of yet further illustration, and referring to U.S. Pat. No. 4,581,028 (the entire disclosure of which is hereby incorporated by reference into this specification), the polymeric material may be selected from the group consisting of polyester (such as Dacron), polytetrafluoroethylene, polyurethane silicone-based material, and polyamide. The polymeric material of this patent is comprised “ . . . of at least one antimicrobial agent selected from the group consisting of the metal salts of sulfonamides.” In one embodiment, the polymeric material is comprised of an antimicrobial agent.

By way of yet further illustration, and referring to U.S. Pat. No. 4,481,353, (the entire disclosure of which is hereby incorporated by reference into this specification), the polymeric material may be the bioresorbable polyester disclosed in such patent.

By way of yet further illustration, and referring to U.S. Pat. No. 4,846,844 (the entire disclosure of which is hereby incorporated by reference into this specification), the polymeric material may be a silicone polymer matrix in which an anabolic agent (such as an anabolic steroid, or estradiol) is disposed.

By way of yet further illustration, and referring to U.S. Pat. No. 4,916,193 (the entire disclosure of which is hereby incorporated by reference into this specification), the polymeric material may be a copolymer containing carbonate repeat units and ester repeat units (see, e.g., claim 1 of the patent). As disclosed in column 2 of the patent, it may also be “collagen,” “homopolymers and copolymers of glycolic acid and lactic acid,” “alpha-hydroxy carboxylic acids in conjunction with Krebs cycle dicarboxylic acids and aliphatic diols,” “polycarbonate-containing polymers,” and “high molecular weight fiber-forming crystalline copolymers of lactide and glycolide.

By way of further illustration, and referring to U.S. Pat. No. 5,176,907 (the entire disclosure of which is hereby incorporated by reference into this specification), the polymeric material may be the poly-phosphoester-urethane described therein. Furthermore, the polymeric material may be one or more of the biodegradable polymers discussed in columns 1 and 2 of such patent.

U.S. Pat. No. 5,176,907 also discloses “In its simplest form, a biodegradable therapeutic agent delivery system consist of a dispersion of the drug solutes in a polymer matrix. The therapeutic agent is released as the polymeric matrix decomposes, or biodegrades into soluble products which are excreted from the body. The “therapeutic agent” used in this (and other) patents may be the anti-mitotic compound of this invention.

By way of yet further illustration, and referring to U.S. Pat. No. 5,194,581 (the entire disclosure of which is hereby incorporated by reference into this specification), the polymeric material may the poly (phosphoester) compositions described in such patent.

The polymeric material may be in the form of microcapsules within which the anti-mitotic compound of this invention is disposed. Thus, one may use microcapsules such as, e.g., the microcapsule described in U.S. Pat. No. 6,117,455, the entire disclosure of which is hereby incorporated by reference into this specification. As is disclosed in the abstract of this patent, there is provided a sustained-release microcapsule contains an amorphous water-soluble pharmaceutical agent having a particle size of from 1 nm-10 μm and a polymer. The microcapsule is produced by dispersing, in an aqueous phase, a dispersion of from 0.001-90% (w/w) of an amorphous water-soluble pharmaceutical agent in a solution of a polymer having a wt. avg. molecular weight of 2,000-800,000 in an organic solvent to prepare an s/o/w emulsion and subjecting the emulsion to in-water drying.

In one embodiment, disclosed in U.S. Pat. No. 5,484,584 (the entire disclosure of which is hereby incorporated by reference into this specification), a poly (benzyl-L-glutamate) microsphere is disclosed (see, e.g., claim 10); the anti-mitotic compound of this invention may be disposed within and/or on the surface of such microsphere. The invention also includes microcapsules surface modified with hydroxyl groups. Various agents such as estrone may be attached to the microcapsules and effectively targeted to selected organs.

The release rate of the anti-mitotic compound from the polymeric material may be varied in, e.g., the manner suggested in column 6 of U.S. Pat. No. 5,194,581, the entire disclosure of which is hereby incorporated by reference into this specification. By way of yet further illustration, and referring to U.S. Pat. No. 5,252,713 (the entire disclosure of which is hereby incorporated by reference into this specification), the polymeric material may be a polypeptide comprising at least one drug-binding domain that non-covalently binds a drug. The means of identifying and isolating such a polypeptide is described at columns 5-7 of the patent.

By way of yet further illustration, and referring to U.S. Pat. No. 5,420,105 (the entire disclosure of which is hereby incorporated by reference into this specification), the polymeric material may form a conjugate with a ligand. Such conjugate may be a ligand or an anti-ligand/polymeric carrier/drug conjugate comprising a ligand consisting of biotin or an anti-ligand selected from the group consisting of avidin and streptavidin, which ligand or anti-ligand is covalently bound to a polymeric carrier that comprises at least one drug-binding domain derived from a drug-binding protein, and at least one drug non-covalently bound to the polymeric carrier, wherein the polymeric carrier does not comprise an entire drug-binding protein, but is derived from a drug-binding domain of said drug-binding protein which derivative non-covalently binds a drug which is non-covalently bound by an entire naturally occurring drug-binding protein, and wherein the molecular weight of the polymeric carrier is less than about 60,000 daltons, and wherein said drug is selected from the group consisting of an anti-cancer anthracycline antibiotic, cis-platinum, methotrexate, vinblastine, mitoxanthrone ARA-C, 6-mercaptopurine, 6-mercaptoguanosine, mytomycin C and a steroid.

The polymeric material may comprise a reservoir (see U.S. Pat. No. 5,447,724) for the anti-mitotic compound(s). Such a reservoir may be constructed in accordance with the procedure described in U.S. Pat. No. 5,447,724.

By way of yet further illustration, and referring to U.S. Pat. No. 5,464,650 (the entire disclosure of which is hereby incorporated by reference into this specification), the polymeric material may be one or more of the polymeric materials discussed at columns 4 and 5 of such patent.

By way of yet further illustration, and referring to U.S. Pat. No. 5,470,307 (the entire disclosure of which is hereby incorporated by reference into this specification), the polymeric material may a synthetic or natural polymer, such as polyamide, polyester, polyolefin (polypropylene or polyethylene), polyurethane, latex, acrylamide, methacrylate, polyvinylchloride, polysuflone, and the like; see, e.g., column 11 of the patent.

In one embodiment, the polymeric material is bound to the anti-mitotic compound by one or more photosensitive linkers. The process of preparing and binding these photosensitive linkers is described in columns 8-9 of U.S. Pat. No. 5,470,307.

In the process of U.S. Pat. No. 5,470,307, the linker is preferably bound to the polymeric material through a modified functional group. The preparation of such modified functional groups is discussed at columns 10-13 of such patent.

As is also disclosed in U.S. Pat. No. 5,470,307, “Acrylic acid can be polymerized onto latex, polypropylene, polysulfone, and polyethylene terephthalate (PET) surfaces by plasma treatment.

By way of yet further illustration, and referring to U.S. Pat. No. 5,599,352 (the entire disclosure of which is hereby incorporated by reference into this specification), the polymeric material can comprise fibrin.

By way of yet further illustration, and referring to U.S. Pat. No. 5,605,696, the polymeric material can be a multi-layered polymeric material, and/or a porous polymeric material.

By way of yet further illustration, and referring to U.S. Pat. No. 5,700,286 (the entire disclosure of which is hereby incorporated by reference into this specification), the polymeric material may be either a thermoplastic or an elastomeric polymer.

By way of yet further illustration, and referring to U.S. Pat. No. 6,004,346 (the entire disclosure of which is hereby incorporated by reference into this specification), the polymeric material may be a bioabsorbable polymer.

As is also disclosed in U.S. Pat. No. 6,004,346, “Several polymeric compounds that are known to be bioabsorbable and hypothetically have the ability to be drug impregnated may be useful in prosthesis formation herein. These compounds include: poly-1-lactic acid/polyglycolic acid, polyanhydride, and polyphosphate ester.

By way of further illustration, and referring to U.S. Pat. No. 6,120,536 (the entire disclosure of which is hereby incorporated by reference into this specification), the polymeric material may comprise a hydrophobic elastomeric material incorporating an amount of anti-mitotic compound therein for timed release. Some of these elastomeric materials are described at columns 5 and 6 of such patent.

As is also disclosed in U.S. Pat. No. 6,120,536, polymers generally suitable for the undercoats or underlayers include silicones (e.g., polysiloxanes and substituted polysiloxanes), polyurethanes, thermoplastic elastomers in general, ethylene vinyl acetate copolymers, polyolefin elastomers, polyamide elastomers, and EPDM rubbers. The above-referenced materials are considered hydrophobic with respect to the contemplated environment of the invention. Surface layer materials include fluorosilicones and polyethylene glycol (PEG), polysaccharides, phospholipids, and combinations of the foregoing.

By way of yet further illustration, and referring to U.S. Pat. No. 6,159,488 (the entire disclosure of which is hereby incorporated by reference into this specification), the polymeric material may be a biopolymer that is non-degradable and is insoluble in biological mediums.

By way of yet further illustration, and referring to U.S. Pat. No. 6,168,801 (the entire disclosure of which is hereby incorporated by reference into this specification), the polymeric material may comprise a material for delivering a biologically active compound comprising a solid carrier material having dissolved and/or dispersed therein at least two biologically active compounds, each of said at least two biologically active compounds having a biologically active nucleus which is common to each of the biologically active compounds, and the at least two biologically active compounds having maximum solubility levels in a single solvent which differ from each other by at least 10% by weight; wherein said solid carrier comprises a biocompatible polymeric material.

The device of U.S. Pat. No. 6,168,801 preferably comprises at least two forms of a biologically active ingredient in a single polymeric matrix. The combination of the at least two forms of the biologically active ingredient or medically active ingredient in at least a single polymeric carrier can provide release of the active ingredient nucleus common to the at least two forms. The release of the active nucleus can be accomplished by, for example, enzymatic hydrolysis of the forms upon release from the carrier device. Further, the combination of the at least two forms of the biologically active ingredient or medically active ingredient in at least a single polymeric carrier can provide net active ingredient release characterized by the at least simple combination of the two matrix forms described above.

By way of yet further illustration, and referring to U.S. Pat. No. 6,395,300 (the entire disclosure of which is hereby incorporated by reference into this specification), the polymeric material may be a porous polymeric matrix.

The anti-mitotic compound may be derived from an anti-microtubule agent. Exemplary microtubule agents are disclosed in U.S. Pat. No. 6,689,803 (at columns 5-6). The anti-microtubule agent may be formulated to further comprise a polymer.

The term “anti-microtubule,” as used herein refers to any “ . . . protein, peptide, chemical, or other molecule which impairs the function of microtubules, for example, through the prevention or stabilization of polymerization. A wide variety of methods may be utilized to determine the anti-microtubule activity of a particular compound, including for example, assays described by Smith et al. (Cancer Lett 79(2):213-219, 1994) and Mooberry et al., (Cancer Lett. 96(2):261-266, 1995);” see, e.g., lines 13-21 of column 14 of U.S. Pat. No. 6,689,803.

An extensive listing of anti-microtubule agents is provided in columns 14, 15, 16, and 17 of U.S. Pat. No. 6,689,803; and one or more of them may be disposed within the polymeric material together with and/or instead of the anti-mitotic compound of this invention. In one embodiment, these prior art anti-microtubule agents are made magnetic in accordance with the process described earlier in this specification.

U.S. Pat. No. 6,689,803 also discloses at columns 16 and 17 that, “Within one preferred embodiment of the invention, the therapeutic agent is paclitaxel, a compound which disrupts microtubule formation by binding to tubulin to form abnormal mitotic spindles. Briefly, paclitaxel is a highly derivatized diterpenoid (Wani et al., J. Am. Chem. Soc. 93:2325, 1971) which has been obtained from the harvested and dried bark of Taxus brevifolia (Pacific Yew) and Taxomyces Andreanae and Endophytic Fungus of the Pacific Yew (Stierle et al., Science 60:214-216,-1993). “Paclitaxel” (which should be understood herein to include prodrugs, analogues and derivatives such as, for example, TAXOL®, TAXOTERE®, Docetaxel, 10-desacetyl analogues of paclitaxel and 3′N-desbenzoyl-3′N-t-butoxy carbonyl analogues of paclitaxel) may be readily prepared utilizing techniques known to those skilled in the art (see e.g., Schiff et al., Nature 277:665-667, 1979; Long and Fairchild, Cancer Research 54:4355-4361, 1994; Ringel and Horwitz, J. Natl. Cancer Inst. 83(4):288-291, 1991; Pazdur et al., Cancer Treat. Rev. 19(4):351-386, 1993; WO 94/07882; WO 94/07881; WO 94/07880; WO 94/07876; WO 93/23555; WO 93/10076; WO94/00156; WO 93/24476; EP 590267; WO 94/20089; U.S. Pat. Nos. 5,294,637; 5,283,253; 5,279,949; 5,274,137; 5,202,448; 5,200,534; 5,229,529; 5,254,580; 5,412,092; 5,395,850; 5,380,751; 5,350,866; 4,857,653; 5,272,171; 5,411,984; 5,248,796; 5,248,796; 5,422,364; 5,300,638; 5,294,637; 5,362,831; 5,440,056; 4,814,470; 5,278,324; 5,352,805; 5,411,984; 5,059,699; 4,942,184; Tetrahedron Letters 35(52):9709-9712, 1994; J. Med. Chem. 35:4230-4237, 1992; J. Med. Chem. 34:992-998, 1991; J. Natural Prod. 57(10):1404-1410, 1994; J. Natural Prod. 57(11):1580-1583, 1994; J. Am. Chem. Soc. 110:6558-6560, 1988), or obtained from a variety of commercial sources, including for example, Sigma Chemical Co., St. Louis, Mo. (T7402—from Taxus brevifolia).”

As is also disclosed in U.S. Pat. No. 6,689,803, “Representative examples of such paclitaxel derivatives or analogues include 7-deoxy-docetaxol, 7,8-cyclopropataxanes, N-substituted 2-azetidones, 6,7-epoxy paclitaxels, 6,7-modified paclitaxels, 10-desacetoxytaxol, 10-deacetyltaxol (from 10-deacetylbaccatin III), phosphonooxy and carbonate derivatives of taxol, taxol 2′,7-di(sodium 1,2-benzenedicarboxylate, 10-desacetoxy-11,12-dihydrotaxol-10,12(18)-diene derivatives, 10-desacetoxytaxol, Protaxol(2′- and/or 7-O-ester derivatives), (2′- and/or 7-O-carbonate derivatives), asymmetric synthesis of taxol side chain, fluoro taxols, 9-deoxotaxane, (13-acetyl-9-deoxobaccatine III, 9-deoxotaxol, 7-deoxy-9-deoxotaxol, 10-desacetoxy-7-deoxy-9-deoxotaxol, Derivatives containing hydrogen or acetyl group and a hydroxy and tert-butoxycarbonylamino, sulfonated 2′-acryloyltaxol and sulfonated 2′-O-acyl acid taxol derivatives, succinyltaxol, 2′-.gamma.-aminobutyryltaxol formate, 2′-acetyl taxol, 7-acetyl taxol, 7-glycine carbamate taxol, 2′-OH-7-PEG(5000)carbamate taxol, 2′-benzoyl and 2′,7-dibenzoyl taxol derivatives, other prodrugs (2′-acetyl taxol; 2′,7-diacetyltaxol; 2′succinyltaxol; 2′-(beta-alanyl)-taxol); 2′gamma-aminobutyryltaxol formate; ethylene glycol derivatives of 2′-succinyltaxol; 2′-glutaryltaxol; 2′-(N,N-dimethylglycyl)taxol; 2′-(2-(N,N-dimethylamino)propionyl)taxol; 2′orthocarboxybenzoyl taxol; 2′aliphatic carboxylic acid derivatives of taxol, Prodrugs {2′(N,N-diethylaminopropionyl)taxol, 2′(N,N-dimethylglycyl)taxol, 7(N,N-dimethylglycyl)taxol, 2′,7-di-(N,N-dimethylglycyl)taxol, 7(N,N-diethylaminopropionyl)taxol, 2′,7-di(N,N-diethylaminopropionyl)taxol, 2′-(L-glycyl)taxol, 7-(L-glycyl)taxol, 2′,7-di(L-glycyl)taxol, 2′-(L-alanyl)taxol, 7-(L-alanyl)taxol, 2′,7-di(L-alanyl)taxol, 2′-(L-leucyl)taxol, 7-(L-leucyl)taxol, 2′,7-di(L-leucyl)taxol, 2′-(L-isoleucyl)taxol, 7-(L-isoleucyl)taxol, 2′,7-di(L-isoleucyl)taxol, 2′-(L-valyl)taxol, 7-(L-valyl)taxol, 2′7-di(L-valyl)taxol, 2′-(L-phenylalanyl)taxol, 7-(L-phenylalanyl)taxol, 2′,7-di(L-phenylalanyl)taxol, 2′-(L-prolyl)taxol, 7-(L-prolyl)taxol, 2′,7-di(L-prolyl)taxol, 2′-(L-lysyl)taxol, 7-(L-lysyl)taxol, 2′,7-di(L-lysyl)taxol, 2′-(L-glutamyl)taxol, 7-(L-glutamyl)taxol, 2′,7-di(L-glutamyl)taxol, 2′-(L-arginyl)taxol, 7-(L-arginyl)taxol, 2′,7-di(L-arginyl)taxol}, Taxol analogs with modified phenylisoserine side chains, taxotere, (N-debenzoyl-N-tert-(butoxycaronyl)-10-deacetyltaxol, and taxanes (e.g., baccatin III, cephalomannine, 10-deacetylbaccatin III, brevifoliol, yunantaxusin and taxusin).”

At columns 17, 18, 19, and 20 of U.S. Pat. No. 6,689,803, several “polymeric carriers” are described. One or more of these “polymeric carriers” may be used as the polymeric material. Thus, and referring to columns 17-20 of such United States patent No., “ . . . a wide variety of polymeric carriers may be utilized to contain and/or deliver one or more of the therapeutic agents discussed above, including for example both biodegradable and non-biodegradable compositions. Representative examples of biodegradable compositions include albumin, collagen, gelatin, hyaluronic acid, starch, cellulose (methylcellulose, hydroxypropylcellulose, hydroxypropylmethylcellulose, hydroxyethylcellulose, carboxymethylcellulose, cellulose acetate phthalate, cellulose acetate succinate, hydroxypropylmethylcellulose phthalate), casein, dextrans, polysaccharides, fibrinogen, poly(D,L lactide), poly(D,L-lactide-co-glycolide), poly(glycolide), poly(hydroxybutyrate), poly(alkylcarbonate) and poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids) and their copolymers (see generally, Illum, L., Davids, S. S. (eds.) “Polymers in Controlled Drug Delivery” Wright, Bristol, 1987; Arshady, J. Controlled Release 17:1-22, 1991; Pitt, Int. J. Phar. 59:173-196, 1990; Holland et al., J. Controlled Release 4:155-0180, 1986). Representative examples of nondegradable polymers include poly(ethylene-vinyl acetate) (“EVA”) copolymers, silicone rubber, acrylic polymers (polyacrylic acid, polymethylacrylic acid, polymethylmethacrylate, polyalkylcynoacrylate), polyethylene, polyproplene, polyamides (nylon 6,6), polyurethane, poly(ester urethanes), poly(ether urethanes), poly(ester-urea), polyethers (poly(ethylene oxide), poly(propylene oxide), Pluronics and poly(tetramethylene glycol)), silicone rubbers and vinyl polymers (polyvinylpyrrolidone, poly(vinyl alcohol), poly(vinyl acetate phthalate). Polymers may also be developed which are either anionic (e.g. alginate, carrageenin, carboxymethyl cellulose and poly(acrylic acid), or cationic (e.g., chitosan, poly-L-lysine, polyethylenimine, and poly (allyl amine)) (see generally, Dunn et al., J. Applied Polymer Sci. 50:353-365, 1993; Cascone et al., J. Materials Sci.: Materials in Medicine 5:770-774, 1994; Shiraishi et al., Biol. Pharm. Bull. 16(11):1164-1168, 1993; Thacharodi and Rao, Int'l J. Pharm. 120:115-118, 1995; Miyazaki et al., Int'l J. Pharm. 118:257-263, 1995). Particularly preferred polymeric carriers include poly(ethylenevinyl acetate), poly (D,L-lactic acid) oligomers and polymers, poly (L-lactic acid) oligomers and polymers, poly (glycolic acid), copolymers of lactic acid and glycolic acid, poly (caprolactone), poly (valerolactone), polyanhydrides, copolymers of poly (caprolactone) or poly (lactic acid) with a polyethylene glycol (e.g., MePEG), and blends thereof.”

As is also disclosed in U.S. Pat. No. 6,689,893, “Polymeric carriers can be fashioned in a variety of forms, with desired release characteristics and/or with specific desired properties. For example, polymeric carriers may be fashioned to release a anti-mitotic compound upon exposure to a specific triggering event such as pH (see e.g., Heller et al., “Chemically Self-Regulated Drug Delivery Systems,” in Polymers in Medicine III, Elsevier Science Publishers B. V., Amsterdam, 1988, pp. 175-188; Kang et al., J. Applied Polymer Sci. 48:343-354, 1993; Dong et al., J. Controlled Release 19:171-178, 1992; Dong and Hoffmann, J. Controlled Release 15:141-152, 1991; Kim et al., J. Controlled Release 28:143-152, 1994; Cornejo-Bravo et al., J. Controlled Release 33:223-229, 1995; Wu and Lee, Pharm. Res. 10(10): 1544-1547, 1993; Serres et al., Pharm. Res. 13(2): 196-201, 1996; Peppas, “Fundamentals of pH- and Temperature-Sensitive Delivery Systems,” in Gumy et al. (eds.), Pulsatile Drug Delivery, Wissenschaftliche Verlagsgesellschaft mbH, Stuttgart, 1993, pp. 41-55; Doelker, “Cellulose Derivatives,” 1993, in Peppas and Langer (eds.), Biopolymers I, Springer-Verlag, Berlin). Representative examples of pH-sensitive polymers include poly(acrylic acid) and its derivatives (including for example, homopolymers such as poly(aminocarboxylic acid); poly(acrylic acid); poly(methyl acrylic acid), copolymers of such homopolymers, and copolymers of poly(acrylic acid) and acrylmonomers such as those discussed above. Other pH sensitive polymers include polysaccharides such as cellulose acetate phthalate; hydroxypropylmethylcellulose phthalate; hydroxypropylmethylcellulose acetate succinate; cellulose acetate trimellilate; and chitosan. Yet other pH sensitive polymers include any mixture of a pH sensitive polymer and a water soluble polymer.”

As is also disclosed in U.S. Pat. No. 6,689,893, “Likewise, polymeric carriers can be fashioned which are temperature sensitive (see e.g., Chen et al., “Novel Hydrogels of a Temperature-Sensitive Pluronic Grafted to a Bioadhesive Polyacrylic Acid Backbone for Vaginal Drug Delivery,” in Proceed. Intern. Symp. Control. Rel. Bioact. Mater. 22:167-168, Controlled Release Society, Inc., 1995; Okano, “Molecular Design of Stimuli-Responsive Hydrogels for Temporal Controlled Drug Delivery,” in Proceed. Intern. Symp. Control. Rel. Bioact. Mater. 22:111-112, Controlled Release Society, Inc., 1995; Johnston et al., Pharm. Res. 9(3):425-433, 1992; Tung, Int'l J. Pharm. 107:85-90, 1994; Harsh and Gehrke, J. Controlled Release 17:175-186, 1991; Bae et al., Pharm. Res. 8(4):531-537, 1991; Dinarvand and D'Emanuele, J. Controlled Release 36:221-227, 1995; Yu and Grainger, “Novel Thermo-sensitive Amphiphilic Gels: Poly N-isopropylacrylamide-co-sodium acrylate-co-n-N-alkylacrylamide Network Synthesis and Physicochemical Characterization,” Dept. of Chemical & Biological Sci., Oregon Graduate Institute of Science & Technology, Beaverton, Oreg., pp. 820-821; Zhou and Smid, “Physical Hydrogels of Associative Star Polymers,” Polymer Research Institute, Dept. of Chemistry, College of Environmental Science and Forestry, State Univ. of New York, Syracuse, N.Y., pp. 822-823; Hoffman et al., “Characterizing Pore Sizes and Water ‘Structure’ in Stimuli-Responsive Hydrogels,” Center for Bioengineering, Univ. of Washington, Seattle, Wash., p. 828; Yu and Grainger, “Thermo-sensitive Swelling Behavior in Crosslinked N-isopropylacrylamide Networks: Cationic, Anionic and Ampholytic Hydrogels,” Dept. of Chemical & Biological Sci., Oregon Graduate Institute of Science & Technology, Beaverton, Oreg., pp. 829-830; Kim et al., Pharm. Res. 9(3):283-290, 1992; Bae et al., Pharm. Res. 8(5):624-628, 1991; Kono et al., J. Controlled Release 30:69-75, 1994; Yoshida et al., J. Controlled Release 32:97-102. 1994; Okano et al., J. Controlled Release 36:125-133, 1995; Chun and Kim, J. Controlled Release 38:39-47, 1996; D'Emanuele and Dinarvand, Int'l J. Pharm. 118:237-242, 1995; Katono et al., J. Controlled Release 16:215-228, 1991; Hoffman, “Thermally Reversible Hydrogels Containing Biologically Active Species,” in Migliaresi et al. (eds.), Polymers in Medicine III, Elsevier Science Publishers B. V., Amsterdam, 1988, pp. 161-167; Hoffman, “Applications of Thermally Reversible Polymers and Hydrogels in Therapeutics and Diagnostics,” in Third International Symposium on Recent Advances in Drug Delivery Systems, Salt Lake City, Utah, Feb. 24-27, 1987, pp. 297-305; Gutowska et al., J. Controlled Release 22:95-104, 1992; Palasis and Gehrke, J. Controlled Release 18:1-12, 1992; Paavola et al., Pharm. Res. 12(12):1997-2002, 1995).”

In one preferred embodiment, the anti-mitotic compound is disposed on or in a drug-eluting polymer that is adapted to elute the anti-mitotic compound at a specified rate. These polymers are well known and are often used in conjunction with drug-eluting stents. Reference may be had, e.g., to U.S. Pat. Nos. 6,702,850 (multi-coated drug-eluting stent), 6,671,562 (high impedance drug eluting cardiac lead), 6,206,914, 6,004,346 (intralumenal drug eluting prosthesis), 5,997,468, 5,871,535 (intralumenal drug eluting prosthesis), 5,851,231, 5,851,217, 5,725,567, 5,697,967 (drug eluting stent), 5,599,352 (method of making a drug eluting stent), 5,591,227 (drug eluting stent), 5,545,208 (intralumenal drug eluting prosthesis), 5,217,028 (bipolar cardiac lead with drug eluting device), 4,953,564 (screw-in drug eluting lead), and the like. The entire disclosure of each of these United States patent Nos. is hereby incorporated by reference into this specification.

A Process for Delivering the Magnetic Anti-Mitotic Compound

FIG. 1 is a schematic of a preferred process 10 for delivering the magnetic anti-mitotic compound described elsewhere in this specification to a specified location. In one embodiment, the magnetic anti-mitotic compound is disposed within a biological organism such as, e.g., a blood vessel 12, and particles 14 of the anti-mitotic compound are delivered to a drug-eluting stent 16.

Referring to FIG. 1, and to the preferred embodiment depicted therein, a bodily fluid, such as blood (not shown for the sake of simplicity of representation) is continuously fed to and through blood vessel 12 in the directions of arrows 20 and 22. In the embodiment depicted, the blood is fed through a generator 26 in order to cause the production of electrical current. In one preferred embodiment, the generator 26 is implanted within an artery 12 or vein 12 of a human being. In another embodiment, not shown, the generator 26 is disposed outside of the artery 12 or vein 12 of the human being.

One may use any of the implanted or implantable generators known to those skilled in the art. Thus, e.g., one may use the power supply disclosed and claimed in U.S. Pat. No. 3,486,506, the entire disclosure of which is hereby incorporated by reference into this specification. This patent claims an electric pulse generator adapted to be implanted within a human body. The generator comprises stator winding means, a permanent magnet rotor rotatably mounted adjacent the stator winding means for inducing electrical potentials therein, and means responsive to the movement of the heart for imparting an oscillatory rotary motion to said rotor at approximately the frequency of the heart beat. In one embodiment, the device of U.S. Pat. No. 3,486,506 is a spring-driven cardiac stimulator.

By way of further illustration, the generator 26 may be the heart-actuated generator described and claimed in U.S. Pat. No. 3,554,199, the entire disclosure of which is hereby incorporated by reference in to this specification.

By way of further illustration, U.S. Pat. No. 3,563,245 (incorporated herein by reference) describes a miniaturized power supply unit that uses the mechanical energy of heart muscle contractions to produce electrical energy for a pacemaker.

The generator 26 may also be the piezoelectric converter disclosed in U.S. Pat. Nos. 3,456,134 and/or 3,659,615 (which are incorporated herein by reference).

By way of yet further illustration, the generator may be that described in U.S. Pat. No. 4,453,537 (incorporated herein by reference), which discloses a pressure actuated artificial heart powered by an implanted device attached to a body muscle; the body muscle is stimulated by an electrical signal from a pacemaker; or that of U.S. Pat. No. 5,810,015 (incorporated herein by reference), which discloses an implantable power supply for converting non-electrical energy to electrical energy.

Referring again to FIG. 1, and to the preferred embodiment depicted therein, the blood preferably flows in the direction of arrow 20, past generator 26, and through stent assembly. The electrical energy from generator 26 is passed via line 28 to regulator 30.

In one embodiment, the generator 26 produces alternating current that is converted into direct current by regulator 30. One may use any of the implantable rectifiers known to those skilled in the art as regulator 30.

Implantable rectifiers are well known and some are described in U.S. Pat. No. 5,999,849 (incorporated herein by reference). As is disclosed in this patent, medical devices that are configured to perform a desired medical function are often implanted in the living tissue of a patient so that a desired function may be carried out as needed for the benefit of the patient.

One may also use the implantable rectifier described in U.S. Pat. No. 6,456,883 (incorporated herein by reference).

Referring again to FIG. 1, and in one preferred embodiment thereof, the regulator 30 is operatively connected to controller 32 by means of a link 34, and the regulator 30 is comprised of an adjustable power supply whose output may be regulated in response to signals fed to such regulator 30 by controller 32.

One may use any of the implantable power supplies known to those in the art as regulator 32. Thus, e.g., one may use the biologically implantable and energized power supply disclosed in U.S. Pat. No. 3,563,245 (incorporated herein by reference).

One may also use the power supply disclosed in U.S. Pat. No. 3,757,795 (incorporated herein by reference).

One may use the power supply disclosed in U.S. Pat. No. 4,143,661 (incorporated herein by reference). The '661 patent describes a power supply system to operate an implanted electric-powered device such as a blood pump. A secondary coil having a biocompatible covering is implanted to subcutaneously encircle either the abdomen or the thigh at a location close to the exterior skin. The secondary coil is electrically interconnected with an implanted storage battery and the blood pump. A primary coil of overlapping width is worn by the patient at a location radially outward of the secondary coil. An external battery plus an inverter circuit in a pack is attached to a belt having a detachable buckle connector which is conventionally worn about the waist. Efficient magnetic coupling is achieved through the use of two air-core windings of relatively large diameter.

One may also use the power supply described in U.S. Pat. No. 4,665,896 (incorporated herein by reference).

By way of yet further illustration, one may use the surgically implanted power supply described in U.S. Pat. No. 5,702,430 (incorporated herein by reference).

By way of yet further illustration, one may use the power supply disclosed in U.S. Pat. No. 5,949,632 (incorporated herein by reference). The '632 patent describes a system whereby power for the internal battery charging circuit is obtained via a subcutaneous secondary coil. This coil is connected to a capacitor/rectifier circuit that is tuned to the carrier frequency being transmitted transcutaneously to the secondary coil. The rectifier may incorporate redundant diodes and a fault detection circuit as shown, which operates similar to the power transistor bridge and logic circuit except that the power transistors are replaced by diodes. This tuned capacitor/rectifier circuit may also incorporate a filter arrangement to support serial communication interface (SCI) reception via the secondary coil. A level detection comparator is provided to convert the analog signal produced by the filter into a digital signal compatible with an SCI receiver. A power transistor or other modulation device may also be incorporated to support SCI transmission via the secondary coil. A redundant transistor bridge such as the bridge used for PWM current limiting may be used in place of the transistor for improved fault tolerance.

Alternatively, one may use the power supply described in U.S. Pat. No. 5,954,058 (incorporated herein by reference). The '058 patent is directed to a rechargeable electrically powered implantable infusion pump and power unit therefor, for intracorporeally dispensing a liquid in a body of a living being, with said infusion pump and power until therefor being capable of subcutaneous implantation in said body of said living being.

One may also use the adjustable power supply described in U.S. Pat. No. 6,141,583 (incorporated herein by reference).

Referring again to FIG. 1, and in the preferred embodiment depicted therein, the generator 26, in one embodiment, produces alternating current This alternating current is fed via line 28 to regulator 30, which preferably converts the alternating current to direct current and either feeds it in a first direction via line 36 to metallic stent 16, or feeds it in another direction via line 38 to metallic stent 16. As will be apparent to those skilled in the art, the regulator 26 thus has the capability of producing a magnetic field of a first polarity (when the direct current is fed in a first direction 36) or a second polarity (when the direct current is fed in a second direction 38), as dictated by the well-known Lenz's law.

In one embodiment, the regulator 26 is capable not only of changing the direction of the electrical current, but also its amount. It can be comprised of a variable resistance circuit that can modulate its output.

In one embodiment, the regulator 26 is comprised of a transceiver (not shown) whose antenna 40 is in telemetric contact with a controller 32. The controller 32 is preferably in telemetric contact with biosensors 42, 44, 46, and/or 48; and, depending upon the information received from one or more of such sensors, can direct the regulator 30 to increase the production of electrical current in one direction, or another, to decrease the production of electrical current in one direction, or another, or to cease the production of electrical current in one direction or another.

Biosensors 42, 44, 46, and/or 48 may be one or more of the implantable biosensors known to those skilled in the art.

In one embodiment, one of such sensors 42, 44, 46, and/or 48 can determine the extent to which two recognition molecules have been bound to each other.

One may use the process and apparatus described in U.S. Pat. No. 5,376,556 (incorporated herein by reference), in which an analyte-mediated ligand binding event is monitored.

By way of further illustration, one may use the “triggered optical sensor” described and claimed in U.S. Pat. No. 6,297,059 (incorporated herein by reference).

Similarly, and by way of further illustration, one may use the light-based sensors discussed at column 1 of U.S. Pat. No. 6,594,011 (incorporated herein by reference).

By way of yet further illustration, one may use one or more of the biological sensors disclosed in U.S. Pat. Nos. 6,546,267 (biological sensor), 5,972,638 (biosensor), 5,854,863, 6,411,834 (biological sensor), 4,513,280 (device for detecting toxicants), 6,666,905, 5,205,292, 4,926,875, 4,947,854 (epicardial multi functional probe), 6,523,392, 6,169,494 (biotelemetry locator), 5,284,146 (removable implanted device), 6,624,940, 6,571,125, 5,971,282, 5,766,934 (chemical and biological sensors having electroactive polymer thin films attached to microfabricated device and possessing immobilized indicator molecules), 6,607,480 (evaluation system for obtaining diagnostic information from the signals and data of medical sensor systems), 6,493,591, 6,445,861, 6,280,586, 5,327,225 (surface plasmon resonance sensor), and the like. The disclosure of each of these United States patents is incorporated herein by reference.

By way of further illustration, one may use the implantable extractable probe described in U.S. Pat. No. 5,205,292 (incorporated herein by reference). This probe comprises a biological sensor attached to the body of the probe such as, e.g., a Doppler transducer for measuring blood flow.

In one embodiment, the nanowire sensor described in published U.S. Patent application no. US20020117659 (incorporated herein by reference) is used.

A drug delivery device that is comprised of a biological sensor is disclosed in published U.S. Patent application US2002/011601 (incorporated herein by reference), which discloses an Implantable Medical Device (IMD) for controllably releasing a biologically-active agent such as a drug to a body. The IMD includes a catheter having one or more ports, each of which is individually controlled by a respective pair of conductive members located in proximity to the port.

At column 1 of published U.S. patent application US2002/0111601, reference is made to other implantable drug delivery systems, any of which might also be used in the present invention. The disclosures of the referenced U.S. Pat. Nos. 5,368,704, 5,797,898, and 5,876,741 are also incorporated herein by reference.

In one embodiment, and referring again to FIG. 1, sensor 36 is an electromagnetic flow meter that, as is known to those skilled in the art, is an instrument which is used to qualitatively measure flow velocity. See, e.g., J. A. Tuszynski et al., “Biomedical Applications of Introductory Physics” (John Wiley & Sons, Inc., New York, N.Y., 2001), page 260.

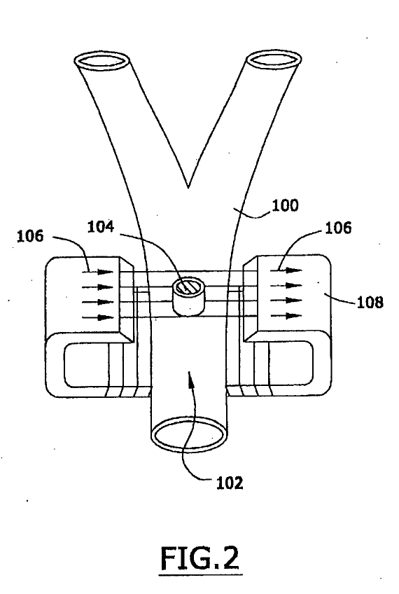

FIG. 2 is a schematic diagram of an electromagnetic flow meter applied to an artery; this Figure is adapted from page 261 of the aforementioned Tuszynski et al. text. Blood (not shown) flows through artery 100 in the direction of arrow 102. A first signal electrode 102 at a first voltage potential is electrically connected to a second signal electrode (not shown) at a second voltage potential. A magnetic field in the direction of arrows is created by magnet 108. As blood flows in the direction of arrow 102 and between the first signal electrode 102 and the second signal electrode (not shown), a current is induced by such flow, and such current is measured by a galvanometer (not shown) that is part of the sensor 36 (see FIG. 1).

In addition to the device depicted in FIG. 2, or instead of such device, one may use one or more of the implantable flow meters known in the art. Thus, e.g., one may use one or more of the implantable flow meters disclosed in U.S. Pat. Nos. 4,915,113 (method and apparatus for monitoring the patency of vascular grants), 6,458,086 (implantable blood flow monitoring system), 6,668,197 (treatment using implantable devices), 6,824,480 (monitoring treatment using implantable telemetric sensors), and the like. The entire disclosure of each of these United States patent Nos. is incorporated herein by reference.

Referring again to FIG. 1, and in the preferred embodiment depicted, a growth of plaque 41 is shown. As will be apparent, and for the sake of simplicity of representation, the plaque 41 is shown on only one portion of the stent 30.

As is known to those in the art, and as is illustrated at page 135 of the Tuszynski et al. text (see problem 11.9), when a segment of an artery is narrowed down by arteriosclerotic plaque to one fifth of its cross-sectional area, the velocity increases five times; but the blood pressure increases about 1 percent.

If one were to use the flow-meter depicted in FIG. 2, and assuming a magnetic field of about 10 Gauss, a blood flow rate of about 20 centimeters per second, a diameter of the artery 100 of about 1 centimeter, the voltage difference between the first electrode 104 and the second electrode (not shown) will be about 1.5 millivolts; and the current flow will be proportional to the resistance in the circuit formed by the two electrodes. With, e.g., a 5 ohm resistance, the current would be about 0.3 milliamperes.

Referring again to FIG. 1, when such current of about 0.3 milliamperes is detected by the sensor 42, such information is preferably transmitted by such sensor 42 to the controller 32. The controller 32 then can determine, based upon this information and other information, to what extent, if any, it wishes to change the activity of regulator 30.

Referring again to FIG. 1, and in the embodiment depicted, the stent 16 also is preferably comprised of sensors 44, 46, and 48. One or more of these sensors may be adapted to detect the amount of anti-mitotic agent in the bloodstream.

Referring again to FIG. 1, and to the preferred embodiment depicted therein, particles of magnetic anti-mitotic agent 14 are fed into the artery 11 by means of source 50. These magnetic particles are directed by an externally applied magnetic field 52 towards the stent 16. As will be apparent, the stent 16 will also have a magnetic moment, depending upon the direction in which current is fed from regulator 30 to the stent 16. When the magnetic moment of the stent is opposite to that of the magnetic anti-mitotic particles 14, the anti-mitotic particles are attracted to the stent 16; when the magnetic moment of the stent 16 is the same as that of the anti-mitotic particles 14, the anti-mitotic particles are directed to the stent. Thus, the controller 32 can control the extent to which, if any, the stent 16 attracts and/or repels the magnetic anti-mitotic particles in its vicinity.

Similarly, when externally applied magnetic field 52 has a magnetic moment that is opposite to that of the magnetic particles, these particles can be driven towards the stent; and they can be pulled from the stent when the externally applied magnetic field has an opposite orientation.

Thus, there are two separate factors that can be varied to either draw the magnetic anti-mitotic particles towards the stent, or to repel such anti-mitotic particles from the stent: the strength and orientation of the magnetic field of the stent (which is controllable via regulator 30), and the strength and orientation of the externally applied magnetic field 52.

One may use any of prior art means for externally applying magnetic field 52. Thus, and referring to published U.S. Patent application no. 2004/0030379 (incorporated herein by reference), an external electromagnetic source or field may be applied to the patient having an implanted coated medical device using any method known to the skilled artisan. For example, the electromagnetic field may be oscillated. Examples of devices which can be used for applying an electromagnetic field include a magnetic resonance imaging (“MRI”) apparatus. Generally, the magnetic field strength suitable is within the range of about 0.50 to about 5 Tesla (Webber per square meter). The duration of the application may be determined based on various factors including the strength of the magnetic field, the magnetic substance contained in the magnetic particles, the size of the particles, the material and thickness of the coating, the location of the particles within the coating, and desired releasing rate of the biologically active material.

Published U.S. Patent application 2004/0030379 also discloses that “In an MRI system, an electromagnetic field is uniformly applied to an object under inspection. At the same time, a gradient magnetic field, superposing the electromagnetic field, is applied to the same. With the application of these electromagnetic fields, the object is applied with a selective excitation pulse of an electromagnetic wave with a resonance frequency which corresponds to the electromagnetic field of a specific atomic nucleus. As a result, a magnetic resonance (MR) is selectively excited. A signal generated is detected as an MR signal. See U.S. Pat. No. 4,115,730 to Mansfield, U.S. Pat. No. 4,297,637 to Crooks et al., and U.S. Pat. No. 4,845,430 to Nakagayashi. The MRI apparatus can be used to create an electromagnetic field. The implanted medical device can be located as is usually done for MRI imaging, and then an electromagnetic field is created by the MRI apparatus to facilitate release of the biologically active material. The duration of the procedure depends on many factors, including the desired releasing rate and the location of the inserted medical device. One skilled in the art can determine the proper cycle of the electromagnetic field, proper intensity of the electromagnetic field, and time to be applied in each specific case.

Referring again to FIG. 1, and in the preferred embodiment depicted therein, in the embodiment depicted, a layer of drug eluting polymer 49 is present in the stent assembly; and this polymer may be used to either attract anti-mitotic agent into it, and/or to elute anti-mitotic agent out of it.

In one preferred embodiment, direct current electrical energy is delivered via lines 36/38 to stent assembly 16. In this embodiment, it is preferred that stent assembly 16 be comprised of conductive material, and that the stent also be comprised of wire-like struts (See, e.g., FIG. 1 of published U.S. Patent application no. 1004/0030379).

As the direct current flows through the conductive material, it creates a static magnetic field in accordance with the well-known Lenz's law. In one embodiment, with the blood flow that is typical through the blood vessels of human beings, magnetic fields on the order of about 1 Gauss can readily be created.

Referring again to FIG. 1, the stent assembly 16 is preferably comprised of a metallic stent body 16 and, disposed thereon, drug eluting polymer 49. The hydrodynamic forces caused by the flow of blood through the stent assembly 16 causes elution of particles 14 of anti-mitotic agent.

It is preferred that regulator 30 be comprised of either a half wave or a full wave rectifier so that the current flowing from regulator 30 be direct current, i.e., that such current flow in only one direction. As will be apparent with either “half-wave d.c.” and/or “full-wave d.c.” being fed to the stent 16, a magnetic field will be induced in such stent that will have a constant polarity but constantly varying intensity. Such a magnetic field will either consistently attract and/or repel the magnetic anti-mitotic particles 14, depending upon the magnetic polarity of such particles. In one preferred embodiment, the magnetized stent 16 consistently attracts the magnetic particles 14.

As will be apparent, the regulator is capable of varying the intensity and/or polarity of its output, preferably in response to a signal from the controller 32. The controller 32 is preferably equipped with an antenna 50 which is in telemetric contact with both the regulator 30 and the sensors 42, 44, 46, and 48.

The sensors 42, 44, 46, and 48 may be any of implantable biosensors known to those skilled in the art.

By way of illustration, and referring to U.S. Pat. No. 4,915,113 (incorporated herein by reference), the sensor(s) may be a implantable Doppler flow meter apparatus for monitoring blood flow through a vascular graft.

The sensor(s) may comprise a means for sensing the strength of a magnetic field. As is disclosed in claim 4 of U.S. Pat. No. 5,562,714 (incorporated herein by reference), the sensing means comprises a sensing antenna having an electrical connection through diodes to a power supply so that the Q of said transmitting antenna is regulated by draw down of energy by said sense antenna through said diode connection to said power supply.

Treatment of In Vivo Tumors with High Frequency Energy

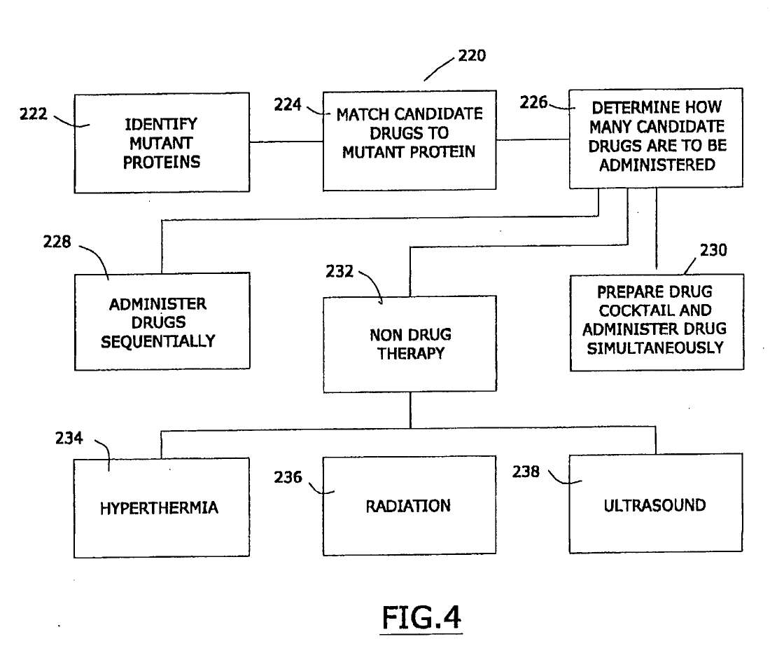

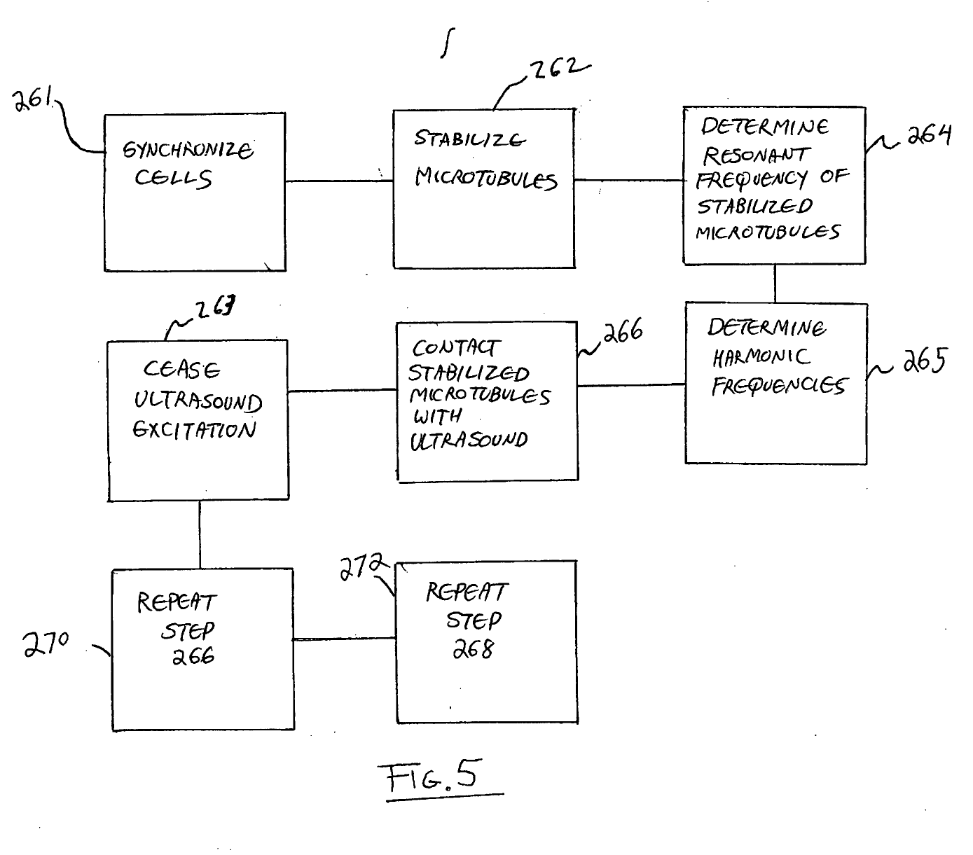

FIG. 5 is a flow diagram of a preferred process 260 for treating a biological organism with mechanical vibrational energy (such as ultrasound) as set forth in step 238 of FIG. 4.

In the process of applicants' invention, in addition to the ultrasound energy, one may use other forms of mechanical energy, some of which are disclosed in published U.S. Patent application no. 2004/0030379 (incorporated herein by reference).

Examples of suitable ultrasound energy are disclosed in U.S. Pat. No. 6,001,069 to Tachibana et al. and U.S. Pat. No. 5,725,494 to Brisken, PCT publications WO00/16704, WO00/18468, WO00/00095, WO00/07508 and WO99/33391, which are all incorporated herein by reference.

Strength and duration of the mechanical vibrational energy may be determined based on various factors including the biologically active material contained in the coating, the thickness of the coating, structure of the coating and desired releasing rate of the biologically active material.

Additional embodiments are identified in published U.S. Patent application no. 2004/0030379: U.S. Pat. Nos. 5,895,356 (a probe for transurethrally applying focused ultrasound energy to produce hyperthermal and thermotherapeutic effect in diseased tissue); 5,873,828 (a device having an ultrasonic vibrator with either a microwave or radio frequency probe); 6,056,735 (an ultrasonic treating device having a probe connected to a ultrasonic transducer and a holding means to clamp a tissue). Any of those methods and devices can be adapted for use in the present invention.

Ultrasound energy application can be conducted percutaneously through small skin incisions. An ultrasonic vibrator or probe can be inserted into a subject's body through a body lumen, such as blood vessels, bronchus, urethral tract, digestive tract, and vagina. However, an ultrasound probe can be appropriately modified, as known in the art, for subcutaneous application. The probe can be positioned closely to an outer surface of the patient body proximal to the inserted medical device.

The duration of the procedure depends on many factors, including the desired releasing rate and the location of the inserted medical device. The procedure may be performed in a surgical suite where the patient can be monitored by imaging equipment. Also, a plurality of probes can be used simultaneously. One skilled in the art can determine the proper cycle of the ultrasound, proper intensity of the ultrasound, and time to be applied in each specific case based on experiments using an animal as a model.

In addition, one skilled in the art can determine the excitation source frequency of the mechanical vibrational energy source. For example, the mechanical vibrational energy source can have an excitation source frequency in the range of about 1 Hertz to about 300 kilohertz. Also, the shape of the frequency can be of different types. For example, the frequency can be in the form of a square pulse, ramp, sawtooth, sine, triangle, or complex. Also, each form can have a varying duty cycle.

Referring to FIG. 5, and in step 261 thereof, the cells of a biological organism to be treated are first preferably synchronized so that they are experiencing substantially synchronous growth. In one aspect of this invention, such cells are synchronized in metaphase.

As is known to those skilled in the art, synchronous growth is growth in which all (or a substantial portion) of the cells are at the same stage of cell division at a given time; this is also often referred to as “synchronized growth.” See, e.g., J. Stensch's “Dictionary of Biochemistry and Molecular Biology,” Second Edition, p. 471 (John Wiley & Sons, New York, 1989); and U.S. Pat. No. 5,18,887, the entire disclosure of which is hereby incorporated by reference into this specification.

In one embodiment, and referring again to FIG. 5, in step 261 the cells of biological organisms are synchronized by means of cell cycle arresting drugs. These drugs are well known to those skilled in the art. See, e.g., European patent publication EP 0 870 506, “Compositions comprising a cryptophytic compound in combination with a synchronizing or activating agent for treating cancer.” The term “synchronizing agent” refers to an agent that can partially synchronize tumor cells with respect to cell cycle progression. Thus the term shall refer to cell cycle phase specific agents such as Gemcitabine, which is now commercially available, and other agents such as multitargeted antifolate (MTA, LY231514), the sulfonylurea LY295501, cisplatin, carboplatin, cyclophosphamide, topoisomerase inhibitor, CPT-11, etoposide, VP-16, 5-fluorouracil, doxorubicin, methotrexate, hydroxyurea and 3′-azido-3′-deoxythymidine (AZT).

Methods for preparing Gemcitabine are known to the skilled artisan and are described in U.S. Pat. No. 4,808,614, herein incorporated by reference in its entirety. See also, European Patent number EP122707 (Sep. 16, 1987).

As used herein the term “activating agent” refers to an agent that can activate non-cycling cells so that they enter the cell cycle where they will be sensitive to cytotoxic agents. Examples of activating agents are growth factors, interleukins, and agents which modulate the function of cell cycle regulation which control cell cycle checkpoints and progression through the cell cycle. For example, but not limited to cdc25 phosphatase or p21. (sdil, wafl, cipl). Such growth factors and interleukins are known and readily available to the skilled artisan.

In one preferred embodiment, the synchronizing agent used is preferably an agent that can partially synchronize tumor cells with respect to cell cycle progression and preferably is a cell cycle phase specific agents such as Gemcitabine.

One may utilize externally applied chemotherapeutic agents to synchronize the cells within a biological organism at a certain stage. Thus, e.g., reference may again be had to U.S. Pat. No. 6,511,818, the entire disclosure of which is hereby incorporated by reference into this specification.

Various references describe how to identify agents that synchronize cells at specific portions of the cell cycle. Reference may be had, e.g., to U.S. Pat. Nos. 5,879,889 (cancer drug screen based on cell cycle uncoupling), 5,882,865 (cancer drug screen based on cell cycle uncoupling), 5,888,735 (cancer drug screen based on cell cycle uncoupling), and 5,879,999 (cancer drug screen based on cell cycle uncoupling). The entire disclosure of each of those references is hereby incorporated by reference.

In one embodiment, a drug is used in such step 261 to synchronize the cells in the organism at the M phase (metaphase), also known as “mitosis.” As is known, mitosis is the division of the nucleus of eucharyotic cells which occurs in four stages designated prophase, metaphase, anaphase, and telophase. In one aspect of this embodiment, the drug used in such step 261 synchronizes the cells in prophase. In one aspect of this embodiment, the drug used in such step 261 synchronizes the cells in metaphase. In one aspect of this embodiment, the drug used in such step 261 synchronizes the cells in anaphase. In one aspect of this embodiment, the drug used in such step 261 synchronizes the cells in telophase.

In one embodiment, the drug used in step 261 stabilizes the cells in metaphase. As is known to those skilled in the art, metaphase is the second stage in mitosis, during which the chromosomes arrange themselves in an equatorial region.

In another embodiment, the drug used in step 261 stabilizes the cells in the “S Phase.” Replication of the nuclear DNA usually occupies only a portion of interphase, called the S phase of the cell cycle. The interval between the completion of mitosis and the beginning of DNA synthesis is called the G1 phase. See, e.g., U.S. Pat. Nos. 4,812,394 (flow cytometric measurement of DNA and incorporated nucleoside analogs), 5,633,945 (accuracy in cell mitosis analysis), 5,866,338 (cell cycle checkpoint genes), 6,172,194 (ARF-p19, a novel regulator of the mammalian cell cycle), 6,274,576 (method of dynamic retardation of cell cycle kinetics to potentiate cell damage), 6,455,593 (method of dynamic retardation of cell cycle kinetics to potentiate cell damage), and the like (all of which are incorporated herein by reference).

As used herein, the term “synchronized” means that at least about 30 weight percent of the cells in question are in the desired phase, and preferably, at least about 50 weight percent of the cells in question are in the desired phase. In one embodiment, at least about 70 weight percent of the cells are in the desired phase.

One may determine the extent to which a collection of cells is synchronized by standard flow cytometry techniques. See, e.g., U.S. Pat. No. 4,812,394 (incorporated herein by reference). A broad range of biological and biomedical investigations depends on the ability to distinguish cells that synthesize DNA from those that do not. Oncologists, for example, have devoted substantial effort to establishing correlations between the proportion of human tumor cells synthesizing DNA and treatment prognosis, e.g. Hart et al., Cancer, Vol. 39, pgs. 1603-1617 (1977). Effort has also been devoted to improvement of anticancer therapy with S-phase specific agents by treating when the experimentally determined proportion of tumor cells in S phase is maximal, e.g. Barranco et al., Cancer Research, Vol. 42, pgs. 2894-2898 (1982). In these studies, S-phase cells are usually assumed to be those that appear labeled in autoradiographs prepared immediately after pulse labeling with tritiated thymidine, or those with S-phase DNA content in DNA distributions measured flow cytometrically. Cancer researchers and oncologists have relied heavily on measurements of the proportion of DNA synthesizing cells to determine the cell cycle traverse characteristics of normal and malignant cells. The classical “fraction of labeled mitosis” procedure, Quastler et al., Experimental Cell Research, Vol. 17, pgs. 420-429 (1959), for example, depends on assessment of the frequency of mitotic cells that appear radioactively labeled in autoradiographs of samples taken periodically after labeling with tritiated thymidine. Studies of the cell cycle traverse characteristics of drug-treated cell populations typically require measurement of the amount of tritiated thymidine incorporated by cells in S phase (e.g., by liquid scintillation spectrometry) or determination of the fraction of cells with S-phase DNA content (e.g., by DNA distribution analysis), or both. Pallavicini et al., Cancer Research, Vol. 42, pgs. 3125-3131 (1982).

Studies of mutagen-induced genetic damage that use unscheduled DNA synthesis as an index of damage also rely on the detection of low levels of incorporation of tritiated thymidine. See, e.g. Painter et al., Biochim. Biophys. Acta, vol. 418, pgs. 146-153 (1976).

One may use other analytical techniques to determine the degree to which the cells are synchronized in a specified phase. In one embodiment, the phase-sensitive flow cytometer described in U.S. Pat. No. 5,270,548 is used; the entire disclosure of this United States patent No. is hereby incorporated by reference into this specification.

Referring again to FIG. 5, one may treat the cells with the synchronizing agent for at least about 25 minutes prior to contact with ultrasound in step 266. It is preferred to wait at least about 60 minutes prior to time one contacts the cells with ultrasound. In one embodiment, one waits at least about 4 hours until after first administration of the synchronizing agent until the cells are contacted with ultrasound. In one embodiment, a period of at least about 48 hours is allowed to pass from the initial administration of the synchronizing agent before the cells so synchronized are contacted with the ultrasound energy.

Referring again to FIG. 5, and in step 262 of this process, microtubules in diseased cells are preferably stabilized by one or more conventional means. As is known to those skilled in the art, stabilization of microtubules at metaphase can result in the synchronization of a population of cells at the metaphase checkpoint of the cell division cycle.

One may effectuate such stabilization by using anti-mitotic or other chemical agents known to affect microtubules, or using chemicals that influence proteins that aid in the stabilization of microtubules (e.g. Rho or FAK), or a process of post-translational modification to the tubulin protein, until the half-life of an individual microtubule in the mitotic spindle of a dividing cell is an average of at least 8 minutes, or more than 10 percent of the microtubules in a non-dividing cell have a half-life of more than 8 minutes. One may use standard means for stabilizing the microtubules to this extent. E.g., U.S. Pat. Nos. 5,808,898 (method of stabilizing microtubules); 5,616,608; 6,403,635; 6,414,015 (laulimalide microtubule stabilizing agents); 6,429,232; 6,500,859 (method for treating atherosclerosis or restenosis using microtubule stabilizing agent); 6,660,767 (coumarin compounds as microtubules stabilizing agents); 6,740,751 (methods and compositions for stabilizing microtubules and intermediate filaments); and the like. The entire disclosure of each of these United States patent Nos. is hereby incorporated by reference.

In step 264 of this process, the resonant frequency of the stabilized microtubules in the diseased cells to be treated is determined. As used herein, the term “resonant frequency” is that frequency which, at a power level of 10 milliwatts per square centimeter, a temperature of 37 degrees Celsius, and atmospheric pressure, is sufficient to break at least 50 weight percent of the microtubules in the cell after an exposure time of five (5) minutes. That frequency which breaks the maximum number of microtubules under these conditions is the resonant frequency.

In step 264 of the process depicted in FIG. 5, an estimate of the energy and wavelengths associated with the vibration of microtubules from an external source is conducted. By way of illustration and not limitation, and without being bound to any particular theory, applicants believe that such an estimate may be readily made in accordance with the discussion and the equations presented elsewhere in this specification.

A Theoretical Approach to Estimate the Type of Ultrasound to be Used in the Process 260

Without wishing to be bound to any particular theory, it is believed that the critical force required to break a microtubule can be calculated by the equation:
Fc˜1/L²
which indicates that the critical force is proportional to 1 divided by the square of L. L is the length of the microtubule.

An estimate of the Fc required to buckle a microtubule can be had from the experimentally derived values of flexural rigidity measured for microtubules. For purposes of this example, and not wishing to be bound to this value, we will assign L to the value of 10 micrometers (μm), and Fc to the value of 6 pN.

Again, for the purposes of this example, without wanting to be bound to a single value, the flexural rigidity of the non-taxol stabilized microtubule can be described with the equation:
EI=10.10⁻²⁴ Nm²
For comparison purposes, actin's critical stress can be described for the purposes of this example:
σ_{c}=5 dyne/cm²=0.5 N/m²

Although not wanting to be bound to this value outside of this example, the buckling pressure of a microtubule has been experimentally determined to be 240 dyne/cm²

The cross sectional area of a hollow tube is described, as in Johnathan Howard's Mechanics of Motor Proteins and the Cytoskeleton (Sinauer Press, 2001), on page 101 to be:
A=(π/4)(d₂²−d₁²)=5×10⁻¹⁶ m².
This equation, in which A represents area, can be applied to microtubules as they are a polymer in the shape of a cylinder and the values of d₂ and d₁, in the case of a microtubule, are simply the outer and inner diameters of the cylinder (25 nm and 15 nm, respectively).

Critical force (F_{c}) can be calculated based on this area in the equation:
F_{c}=P_{c}×A=0.4 pN
in which P_{c} represents the critical pressure applied perpendicularly to the cross-sectional area A.

Young's modulus (Y) is a description of the stiffness of a material. Young's modulus for microtubules has been experimentally determined Y=10⁹ N/m².

The spring constant (k) for a microtubule can be calculated from the Young modulus as given below:
k=(A×Y)/L
in which A is the area of the cylindrical cross-section (described above), Y is the Young's modulus and L is the length of the microtubule, therefore:
k=(π(25²−15²)×10⁹)/10=˜4 N/m.

This value is important because it is greater than the force of attraction between 2 protofilaments in a microtubule structure (2 N/m).

In general, one should use the formula (see the book by Jonathon Howard) to derive the formula for the critical force:
F_{c}=π²(EI/L²),
and thus calculate the propagation velocity for a standing vibrational wave in a microtubule by way of the following equation:
υ=(F/ρ_{L})^{1/2}
in which ρ_{L} is the linear mass density of the protein filament (microtubule) and F stands for the tension force that is less or at best equal to the critical force for breaking a microtubule.

One can then calculate the frequency of the vibrational mode according to:
f=v/l=(F/ρ_{L})^{1/2}/l
where l is the wavelength of the standing wave. The fundamental harmonic will have the wavelength l=2L where L is the length of the microtubule cylinder along its axis. In general, the n-th harmonic will have the wavelength given by the formula: lₙ=2L/n. Hence, its frequency is given by: fₙ=nf, where f stands for the fundamental harmonic. The formula above is applied for the calculation of the fundamental harmonic, second harmonic, or third harmonic, etc., by choosing the value of n as 1, 2, 3, etc. For purposes of this example, EI is assigned to be 26×10⁻²⁴ Nm² in its native state while attached at both ends (one to a polar body, the other to a chromosome, as in mitosis). This value increases to 32×10⁻²⁴ Nm² when the microtubule is stabilized with taxol. Using this value, we can estimate the frequency to be in the range of 270-420 kHz for the fundamental harmonic with a second harmonic at twice the frequency to be in the range of 540-840 kHz, etc.

It should be noted that the frequency formula depends inversely proportionally to the length of a given microtubule. In this connection, polar microtubules are almost twice as long as kinetochore microtubules and hence, in order to break them by means of applying high frequency ultrasound, different frequency ranges must be selected (approximately half the values of those applied to break kinetochore microtubules). In general, this application of ultrasound for breaking up the mitotic apparatus in dividing cells requires a prior microscopic observation and analysis of the cell's cytoskeletal apparatus with particular attention to the length of the microtubules to be determined as accurately as possible. Having determined the lengths and elastic constants for all kinetochore and polar microtubules, a weighted superposition of the fundamental and first harmonic ultrasound modes must be calculated and then generated with a subsequent application to the cellular targets.

The mass density of tubulin is estimated to be approximately 900 kg/m³ while that of the surrounding medium (mainly water) is assumed to be 1000 kg/m³. The linear mass density of a microtubule cylinder is calculated assuming the length L, the outer and inner diameters d₂ and d₁, respectively, as stated above. Aqueous environment is filling the inner diameter region of the cylinder as well as forming a thin layer of bound water surrounding the outer surface. We assumed that a 3 angstrom layer of bound water is attached. With these assumptions, the linear mass density (mass per length) of a microtubule is approximately 5×10⁻¹³ kg/m. Using the formula for v stated above as a function of the force of tension applied to a microtubule (at most 6 pN) and the above linear mass density, we evaluate the propagation velocity of standing vibrational waves on microtubules to be in the range of 3-4 m/s which is much less than the propagation velocity of ultrasound in an aqueous medium (on the order of 1000 m/s).

The following is an estimate of the ultrasound intensity required to deliver a sufficiently strong amount of energy to break microtubules. The formula for the power delivered per cross-sectional area for a wave traveling at a speed v in a medium of mass density rho and having an amplitude A is given by: Power/Area=A² v f² rho, where f is the frequency of the wave. Estimating the amplitude A to be in the 3 angstrom range, the frequency in the MHz range and the velocity of propagation as well as mass density as given above, we obtain an estimate of the intensity as 0.1 W/m². However, this is only the power deposited in the form of microtubule oscillations. Since the ultrasound propagates at a much faster velocity in the medium before it is resonantly absorbed by the microtubules, the actual power generated at the source most be scaled up by the velocity ratio factor, i.e. we expect it to be at least in the range of 10-30 W/m² which corresponds to the 130-135 dB range on the decibel scale.

It is known that paclitaxel (Taxol®), and paclitaxel-type compounds, stabilize microtubules, prevent them from shortening and dividing the cell as a result of their shortening as they segregate the genetic material in chromosomes. Furthermore, paclitaxel increases the rigidity of microtubules making them susceptible to breaking given the right physical stimuli.

Ultrasound induces mechanical vibrations of microtubules. At the right frequency, and at the right power level, the application of ultrasound will cause the microtubules to first buckle and then break up.

The ultrasound used in this invention has a frequency of about 10 kHz to about 10 GHz. Alternatively, the frequency can be about 50 megaHz to about 2 GHz. In other embodiments, the frequency is about 100 megaHz to about 1 GHz. The power of such ultrasound will generally be at least about 0.01 watts per square meter. Alternatively, it will be at least about 10 watts per square meter.

The ultrasound is preferably focused on the tumor to be treated. One may use any conventional means for focusing ultrasound. One may use one or more of the devices disclosed in U.S. Pat. Nos. 6,613,0055 (systems and methods for steering a focused ultrasound array), 6,613,004, 6,595,934 (skin rejuvenation using high intensity focused ultrasound), 6,543,272 (calibrating a focused ultrasound array), 6,506,154 (phased array focused ultrasound system), 6,488,639 (high intensity focused ultrasound treatment apparatus), 6,451,013 (tonsil reduction using high intensity focused ultrasound to form an ablated tissue area), 6,432,067 (medical procedures using high-intensity focused ultrasound), 6,425,867 (noise-free real time ultrasonic imaging of a treatment site undergoing high intensity focused ultrasound therapy), and the like. The entire disclosure of each of those references is incorporated by reference into this specification.

In one embodiment, paclitaxel (or a similar composition) is delivered to the patient and, as is its wont, makes the microtubules more rigid. Thereafter, when the microtubules are polymerized in a dividing cell and substantially immobilized, the ultrasound is selectively delivered to the microtubules in the tumor, thereby breaking such microtubules and halting the process of cell growth and division, ultimately leading to cell death (apoptosis).

In one aspect of this embodiment, after the paclitaxel (or similar material) has been delivered to the patient, a high intensity magnetic field is applied to the tumor in order to selectively cause the paclitaxel to bind the microtubules in the tumor. Thereafter, the ultrasound is applied to break the microtubules so bound to the paclitaxel enhancing the efficacy of the drug due to a combined effect of the magnetic field, ultrasound and chemotherapeutic action of paclitaxel itself.

When microtubules have been broken, they tend to reform. Therefore, in one embodiment, and referring again to FIG. 5, the ultrasound is periodically or continuously delivered to the tumor synchronized to the typical time elapsed between subsequent cell division processes during which microtubules are polymerized (see, e.g., steps 261/270/272 of FIG. 5).

In one embodiment, a portable device is worn by the patient and applied to the tumor site; and this device periodically and/or continuously delivers ultrasound and/or magnetic energy to the patient. In one aspect of this embodiment, the device first delivers high intensity magnetic energy, and then it delivers the ultrasound energy. Referring again to FIG. 5, and to the preferred embodiment depicted therein, in step 265 one can determine the harmonic frequencies that correspond to the resonant frequency determined in step 264. One may use a first harmonic of such resonant frequency, a second harmonic of such resonant frequency, and, in fact, any harmonic of the resonant frequency. As is known to those skilled in the art, a harmonic is one of a series of sounds, each of which has a frequency that is an integral multiple of some fundamental frequency.

One may apply the resonant frequency to the stabilized microtubules and/or one of the harmonic frequencies, and/or a second of the harmonic frequencies and/or a third of the harmonic frequencies and/or a fourth of the harmonic frequencies and/or a fifth of the harmonic frequencies, etc. These frequencies may be applied simultaneously, and/or they may be applied sequentially. One may alternate this application of frequency or frequencies with the administration of one or more stabilizing agents and/or synchronizing agents and/or antimitotic agents and/or cytotoxic agents.

In this process, and in step 266 thereof, one may use any of the means for generating and focusing ultrasound energy that are known to those skilled in the art. One may use the ultrasound generator disclosed in U.S. Pat. No. 6,685,639 (incorporated herein by reference).

By way of yet further illustration, and not limitation, one may use one or more of the ultrasound generators described in U.S. Pat. Nos. 3,735,756 (duplex ultrasound generator); 4,718,421 (ultrasound generator); 4,957,100 (ultrasound generator and emitter); 4,976,255 (extracorporeal lithotripsy using shock waves and therapeutic ultrasound); 5,102,534; 5,184,065 (therapeutic ultrasound generator); 5,443,069 (therapeutic ultrasound applicator for the urogenital region); 6,270,342; and the like. The entire disclosure of each of these United States patent Nos. is hereby incorporated by reference into this specification.

By way of further illustration, one may also use the ultrasound generator disclosed in I. Hrazdira et al., “Ultrasonically inducted alterations of cultured tumour cells,” European Journal of Ultrasound 8: 43-49, 1998.

Without wishing to be bound to any particular theory, the resonant frequency will likely vary with the square root of the average length of the microtubules in the cells being treated. The microtubules in diseased cells do not necessarily have the same length as the microtubules in non-diseased cells. Cancer cells have microtubules that are up to about 10 percent longer than the microtubules of comparable non-cancer cells. Thus, by applying frequencies that are specific for the microtubules in the diseased cells, one can preferentially treat the diseased cells with the process of this invention. Moreover, for the ultrasound application to be most effective in breaking up tumor cell microtubules, an appropriate superposition of frequencies must be applied in correspondence to the lengths and rigidities of microtubules targeted.

Referring again to FIG. 5, and to step 264 thereof, a series of experiments may be preferably conducted with ultrasound waves with a power level of 10 milliwatts per square centimeter and different frequencies, at temperature of 37 degrees Celsius, and atmospheric pressure, and then the breakage of microtubules caused by such exposure is determined. That frequency which breaks the maximum number of microtubules is the resonant frequency, as will be apparent, the results of these experiments may be used to corroborate the estimates made by mathematical means of the resonant frequency of the stabilized microtubules. Alternatively, they may be used independently to determine the resonant frequency of the microtubules.

One may determine the extent to which any particular ultrasound wave breaks microtubules by conventional means. One may use the means described in the aforementioned article by I. Hrazdira et al. (“Ultrasonically induced alterations of cultured tumor cells,” European Journal of Ultrasound, 8 [1998], 43-49, Section 2.3). For visualization of cytoskeleton components, an indirect immunofluorescence method was used. The cells in the monolayer were washed with phosphate buffer before adding 0.1% Triton for stabilization of membrane permeability. The cells were subsequently fixed by means of 3% paraformaldeyde. After fixation, secondary antibodies were added for 45 min for microtubules. Between each operation, the cells were washed by PBS. Finally, samples for fluorescence microscopy were prepared. A total of 20 microphotographs of each control and experimental sample were evaluated anonymously. Changes in cytoskeletal structure were evaluated quantitatively.

Referring again to FIG. 5, and in step 266 of the process, the stabilized microtubules are then contacted with ultrasound energy.

In one embodiment, the frequency of the ultrasound energy is approximately the resonant frequency, plus or minus about ten percent. In one aspect of this embodiment, the frequency of the ultrasound energy is approximately the resonant frequency, plus or minus about 5 percent. In general, such frequency will often be in the range of from about 100 kilohertz to about 500 kilohertz. and, more preferably, from about 110 to about 200 kilohertz. In yet another embodiment, such frequency is from about 130 to about 170 kilohertz.

The power used for such exposure is preferably from about 1 to about 30 milliwatts per square centimeter and, more preferably, from about 5 to about 15 milliwatts per square centimeters.

To help insure that applicants' process is more effective in causing permanent changes in the cell, and in step 268, the ultrasound excitation of the stabilized microtubules is ceased when the temperature of such microtubules reaches a specified temperature such as, e.g., a temperature of 70° Celsius.

U.S. Pat. No. 6,685,639, the entire disclosure of which is hereby incorporated by reference, describes a high intensity focused ultrasound system for scanning and treating tumor which creates a very high temperature (in excess of 70° Celsius) in the area of the “focal region.” By means of focusing, the system causes ultrasonic waves to form a space-point with high energy (focal region); the energy of the region reaches over 1000 W/m² and the temperature instantaneously rises to greater than 70° Centigrade.

Applicants wish to avoid prolonged exposure of the cells of living organisms to a temperature in excess of a specified temperature, such as, e.g., 42° Celsius. Thus, when the temperature of the microtubules reaches such specified temperature, the ultrasound excitation of the stabilized microtubules is ceased (step 268), and then the process of ultrasound excitation is repeated.

Thereafter, in step 270, step 266 (the contacting of the stabilized microtubules with ultrasound energy) is repeated until the temperature of the microtubules reaches the aforementioned maximum temperature, at which point step 268 is repeated (in step 272). The cycle is continued for as many times as is necessary to induce apoptosis.

In one embodiment, step 266 is conducted for about 1 to about 5 minutes, the microtubules are allowed to cool, and then step 266 is repeated again and again.

Although preferred embodiments have been depicted and described in detail herein, it will be apparent to those skilled in the relevant art that various modifications, additions, substitutions, and the like can be made without departing from the spirit of the invention, and these are thus considered to be within the scope of the invention as defined in the claims which follow.

What is claimed is:

1. A process for treating a biological organism, comprising the steps of:
(a) administering a cell cycle arresting drug to said organism, thereby producing synchronized cells within such organism,
(b) administering a microtubule stabilizing drug to said organism, thereby producing synchronized cells whose microtubules have been stabilized within said organism, and
(c) contacting said synchronized cells whose microtubules have been stabilized with mechanical vibrational energy.

2. The process as recited in claim 1, wherein said mechanical vibrational energy has an excitation source frequency in the range of from about 1 hertz to about 10 Gigahertz.

3. The process as recited in claim 1, wherein said cell cycle arresting drug synchronizes tumor cells with respect to cell cycle progression.

4. The process as recited in claim 3, wherein said cell cycle arresting drug is selected from the group consisting of gemcitabine, cisplatin, carboplatin, cyclophosphamide, topoisomerase inhibitor, etoposide, 5-fluoroacil, doxorubicin, methotrexate, hydroxyurea, 3′-azido-3′-deoxythymidine, and mixtures thereof.

5. The process as recited in claim 3, wherein said cell cycle arresting drug is gemcitabine.

6. The process as recited in claim 1, wherein said cell cycle arresting drug synchronizes said cells in metaphase.

7. The process as recited in claim 1, wherein said cell cycle arresting drug synchronizes said cells in anaphase.

8. The process as recited in claim 6, wherein at least about 30 percent of said cells are synchronized in metaphase.

9. The process as recited in claim 6, wherein at least about 50 percent of said cells are synchronized in metaphase.

10. The process as recited in claim 6, wherein at least about 70 percent of said cells are synchronized in metaphase.

11. The process as recited in claim 1, wherein said mechanical vibrational energy is ultrasound.

12. The process as recited in claim 11, wherein said synchronized cells are contacted with said ultrasound only after at least 25 minutes after said cell cycle arresting drug has been administered to said organism.

13. The process as recited in claim 11, wherein said synchronized cells are contacted with said ultrasound only after at least 60 minutes after said cell cycle arresting drug has been administered to said organism.

14. The process as recited in claim 11, wherein said synchronized cells are contacted with said ultrasound only after at least 240 minutes after said cell cycle arresting drug has been administered to said organism.

15. The process as recited in claim 11, wherein said synchronized cells are contacted with said ultrasound only after at least 48 hours after said cell cycle arresting drug has been administered to said organism.

16. The process as recited in claim 11, wherein said microtubule stabilizing drug is a laulimalidge microtubule stabilizing agent.

17. The process as recited in claim 11, wherein said microtubule stabilizing drug is a coumarin compound.

18. The process as recited in claim 11, wherein said ultrasound has a frequency of from about 270 to about 420 kilohertz.

19. The process as recited in claim 11, wherein said ultrasound has an intensity of from about 10 to about 30 watts per square meter.

20. The process as recited in claim 11, wherein said microtubule stabilizing drug is paclitaxel.

21. The process as recited in claim 11, wherein said ultrasound has a frequency of from about 50 megahertz to about 2 gigahertz.

22. The process as recited in claim 11, wherein said ultrasound has a frequency of from about 100 megahertz to about 1 gigahertz.

23. The process as recited in claim 11, wherein the power of said ultrasound is at least about 0.01 watts per square meter.

24. The process as recited in claim 11, wherein the power of said ultrasound is at least about 0.1 watts per square meter.

25. The process as recited in claim 11, wherein said ultrasound has a frequency of form about 100 kilohertz to about 500 kilohertz.

26. The process as recited in claim 11, wherein said ultrasound has a frequency of from about 110 to about 200 kilohertz.

27. The process as recited in claim 11, wherein said ultrasound has a frequency of from about 130 to about 170 kilohertz.

28. The process as recited in claim 11, wherein the power of said ultrasound is from about 1 to about 30 watts per square meter.

29. The process as recited in claim 11, wherein the power of said ultrasound is from about 5 to about 15 watts per square meter.

30. A process for initiating apoptosis in a cancer cell comprising:
(a) contacting the cell with a cell cycle arresting drug; and
(b) contacting said cell with mechanical vibrational energy.

31. The process of claim 30, wherein the cell cycle arresting drug is selected from the group consisting of: gemcytabine, cisplatin, carboplatin, cyclophosphamide, topoisomerase inhibitor, etoposide, 5-fluorouracil, doxorubicin, methotrexate, hydroxyurea, and 3′-azido-3′-deoxythymidine.

32. The process of claim 30, wherein the mechanical vibrational energy is ultrasound energy having a frequency of about 50 megahertz to about 2 gigahertz.

33. The process of claim 30, wherein the exposure to mechanical vibrational energy is repeated or sustained over a period of at least one typical cell cycle.

34. The process of claim 30, wherein the step of contacting the cell with mechanical vibrational energy is repeated or sustained over a period of at least one typical cell division.

35. The process of claim 30, further comprising a step of synchronizing tubulin assembly in the cell.

36. The process of claim 35, wherein the step of synchronizing tubulin assembly is effected by exposing the cell to a microtubule stabilizing agent and/or radiation, and the step is performed prior to contacting the cell with mechanical vibrational energy.

37. The process of claim 36, wherein the microtubule stabilizing agent is selected from the group consisting of taxanes, coumarins, and combinations thereof.

38. A method for treating a patient suffering from cancer comprising administering to said patient an amount of a cell cycle arresting drug sufficient to synchronize cell cycles of a plurality of cancer cells in said patient; and subjecting said cells to mechanical vibrational energy.

39. The method of claim 38, wherein the cell cycle arresting drug is selected from the group consisting of: gemcytabine, cisplatin, carboplatin, cyclophosphamide, topoisomerase inhibitor, etoposide, 5-fluorouracil, doxorubicin, methotrexate, hydroxyurea, and 3′-azido-3′-deoxythymidine.

40. The method of claim 38, wherein the mechanical vibrational energy is ultrasound energy having a frequency of about 50 megahertz to about 2 gigahertz.

41. The method of claim 38, wherein the exposure to mechanical vibrational energy is repeated or sustained over a period of at least one typical cell cycle.

42. A method of treating a patient suffering from cancer comprising administering to said patient an amount of a cell cycle arresting drug sufficient to synchronize cell cycles of a plurality of cancer cells in the patient; administering to the patient a microtubule stabilizing agent; and exposing the patient to mechanical vibrational energy.

43. The method of claim 42, wherein the microtubule stabilizing agent is selected from the group consisting of: taxanes, magnetic taxanes; coumarins, magnetic coumarins, and combinations thereof.

44. The method of claim 42, wherein the microtubule stabilizing agent is selected from the group consisting of paclitaxel, docetaxel, magnetic derivatives thereof, and combinations thereof.

45. The method of claim 42, wherein the cell cycle arresting drug is selected from the group consisting of: gemcytabine, cisplatin, carboplatin, cyclophosphamide, topoisomerase inhibitor, etoposide, 5-fluorouracil, doxorubicin, methotrexate, hydroxyurea, and 3′-azido-3′-deoxythymidine.

46. The method of claim 42, wherein the cell cycle arresting drug is gemcytabine and the microtubule stabilizing agent is a taxane, a coumarin, magnetic derivatives thereof, and combinations thereof.

47. The method of claim 42, wherein the mechanical vibrational energy is ultrasound energy having a frequency of about 50 megahertz to about 2 gigahertz.

48. The method of claim 42, wherein exposure to mechanical vibrational energy is repeated or sustained over a period of at least one typical cell cycle.

49. The method of claim 42, wherein exposure to mechanical vibrational energy is performed at least 60 minutes after administration of the cell cycle arresting drug.

50. The method of claim 42, wherein the microtubule stabilizing agent is administered from a drug eluting implant.

51. The method of claim 50, wherein the implant is a drug eluting stent.

52. A process for treating a patient suffering from cancer comprising administering to said patient an amount of a cell cycle arresting drug sufficient to synchronize cell cycles of a plurality of the cancer cells in said patient; administering to said patient radiation therapy sufficient to stabilize microtubule assembly in said cancer cells; and subjecting said cancer cells to mechanical vibrational energy.

53. A method of treating a patient suffering from cancer comprising administering to said patient a cancer cell cycle arresting amount of gemcytabine; administering a microtubule stabilizing agent selected from the group consisting of taxanes, coumarins, magnetic derivatives thereof, and combinations thereof; and exposing the patient to mechanical vibrational energy of frequency of about 50 megahertz to about 2 gigahertz.

54. The method of claim 53, wherein the microtubule stabilizing agent is selected from the group consisting of paclitaxel, docetaxel, magnetic derivatives thereof, and combinations thereof.

55. A method of treating a patient suffering from cancer comprising administering to said patient an amount of a cell cycle arresting drug sufficient to synchronize cell cycles of a plurality of cancer cells in the patient; administering to the patient a magnetic microtubule stabilizing agent; applying a localized magnetic field to increase the concentration of magnetic microtubule stabilizing agent at a predetermined location in the patient; and exposing the patient to mechanical vibrational energy.

56. The method of claim 55, wherein the magnetic microtubule stabilizing agent is a magnetic taxane or a magnetic coumarin.

57. The method of claim 55, wherein the mechanical vibrational energy is ultrasound energy of frequency of about 50 megahertz to about 2 gigahertz.

58. The method of claim 57, wherein the ultrasound energy is administered to the patient by an intracorporeal device.
